(19) United States
(12) Patent Application Publication
(10) Pub. No.: US 2022/0048919 A1
(43) Pub. Date: Feb. 17, 2022

(54) HETEROARYLDIHYDROPYRIMIDINE DERIVATIVES AND METHODS OF TREATING HEPATITIS B INFECTIONS

(71) Applicant: Janssen Phrmaceutica NV, Beerse (BE)

(72) Inventors: Yanping XU, Noblesville, IN (US); Yimin JIANG, Londonberry, NH (US); Wangyang TU, Shanghai (CN); Gang DENG, Shanghai (CN); Zhanling CHENG, Shanghai (CN); Chao LIANG, Shanghai (CN)

(21) Appl. No.: 17/415,696

(22) PCT Filed: Dec. 19, 2019
(86) PCT No.: PCT/CN2019/126735

§ 371 Date: Jun. 17, 2021

Related U.S. Application Data
(60) Provisional application No. 62/791,524, filed on Jan. 11, 2019.

(30) Foreign Application Priority Data
Dec. 20, 2018 (CN) PCT/CN2018/122257

(51) Int. Cl. C07D 487/04; A61K 45/06

(52) U.S. Cl. CPC C07D 487/04; C07D 417/04; A61K 45/06

(57) ABSTRACT

Provided herein are compounds useful for the treatment of HBV infection in a subject in need thereof, pharmaceutical compositions thereof, and methods of inhibiting, suppressing, or preventing HBV infection in the subject.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

There is a need in the art for therapeutic agents that can increase the suppression of virus production and that can treat, ameliorate, or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

Background art on heteroaryldihydropyrimidines for use in the treatment of HBV includes WO 2015/132276, WO2013/102655 and WO99/54326.

### SUMMARY

Provided herein are compounds useful for the treatment of HBV infection in a subject in need thereof. Thus, in an aspect, provided herein is a compound of Formula I

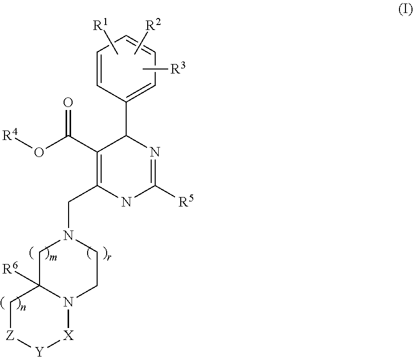

including the deuterated isomers, stereoisomers or tautomeric forms thereof, or a pharmaceutically acceptable salt thereof, wherein:
R¹, R² and R³ are independently selected from the group consisting of H, halogen and C₁₋₄alkyl;
R⁴ is C₁₋₄alkyl;
R⁵ is thiazolyl, or pyridyl optionally substituted with one or more substituents selected from the group consisting of fluorine and C₁₋₃alkyl;
R⁶ is C₁₋₄alkyl, optionally substituted with a substituent selected from the group consisting of OH and CN;
m is 1;
r is 1;
n is an integer of 0 or 1;

### X is C(═O), C(═S), or SO₂;

### Y is NR⁷;

R⁷ is selected from the group consisting of H, —C₁₋₆alkyl, —C₁₋₆alkyl-R⁸, —C₁₋₆alkoxy-C₁₋₆alkyl-R⁸, —(CH₂)ₚ—C(R¹¹R¹²)—R⁸ and —(CH₂)ₚ-Q-R⁸;
R⁸ is selected from the group consisting of —C₁₋₆alkyl, —COOH, —C(═O)NHS(═O)₂—C₁₋₆alkyl, tetrazolyl and carboxylic acid bioisosteres;
R¹¹ and R¹² together with carbon atom to which they are attached form a 3-7 membered saturated ring optionally containing a heteroatom, the heteroatom being an oxygen or a nitrogen substituted with R⁹;
Q is selected from the group consisting of aryl, heteroaryl, and a 3-7 membered saturated ring optionally containing a heteroatom, the heteroatom being an oxygen or a nitrogen substituted with R⁹;
R⁹ is H, C₁₋₆ alkyl, or C₁₋₆ alkoxy-C₁₋₆alkyl;
p is an integer of 0, 1, 2, or 3;

### Z is CH₂ or C(═O).

In another aspect, provided herein is a pharmaceutical composition comprising at least one compound of Formula I, or a pharmaceutically acceptable salt thereof, together with a pharmaceutically acceptable carrier.

In another aspect, provided herein is a pharmaceutical composition comprising at least one disclosed compound, together with a pharmaceutically acceptable carrier.

In another aspect, provided herein is a method of treating an HBV infection or of an HBV-induced disease in an individual in need thereof, comprising administering to the individual a therapeutically effective amount of a compound of Formula I or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula I, or a pharmaceutically acceptable salt thereof.

In an embodiment, any of the methods provided herein can further comprising administering to the individual at least one additional therapeutic agent selected from the group consisting of HBV inhibitors as herein further defined.

### DETAILED DESCRIPTION

Provided herein are compounds, e.g., the compounds of I, or pharmaceutically acceptable salts thereof, that are useful in the treatment and prevention of HBV infection in subject.

Without being bound to any particular mechanism of action, these compounds are believed to modulate or disrupt HBV assembly and other HBV core protein functions necessary for HBV replication or the generation of infectious particles and/or may disrupt HBV capsid assembly leading to empty capsids with greatly reduced infectivity or replication capacity. In other words, the compounds provided herein may act as capsid assembly modulators.

The compounds provided herein have potent antiviral activity, exhibit favorable metabolic properties, tissue distribution, safety and pharmaceutical profiles, and are suitable for use in humans. Disclosed compounds may modulate (e.g., accelerate, delay, inhibit, disrupt or reduce) normal viral capsid assembly or disassembly, bind capsid or alter metabolism of cellular polyproteins and precursors. The modulation may occur when the capsid protein is mature, or during viral infectivity. Disclosed compounds can be used in methods of modulating the activity or properties of HBV cccDNA, or the generation or release of HBV RNA particles from within an infected cell.

In one embodiment, the compounds described herein are suitable for monotherapy and are effective against natural or native HBV strains and against HBV strains resistant to currently known drugs. In another embodiment, the compounds described herein are suitable for use in combination therapy.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity or is lethal to the virus.

As used herein, the term “treatment” or “treating” is defined as the application or administration of a therapeutic agent, i.e., a disclosed compound (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection, or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “alkyl,” by itself or as part of another substituent means, unless otherwise stated, a straight or branched chain hydrocarbon having the number of carbon atoms designated (i.e., C₁-C₃ alkyl means an alkyl having one to three carbon atoms, C₁-C₄ alkyl means an alkyl having one to four carbon) and includes straight and branched chains. Examples include methyl, ethyl, propyl, isopropyl, butyl, isobutyl, tert-butyl. Embodiments of alkyl generally include, but are not limited to, C₁-C₁₀ alkyl, such as C₁-C₆ alkyl, such as C₁-C₄ alkyl.

As used herein, the term “alkenyl,” by itself or as part of another substituent means, unless otherwise stated, a linear or branched chain of hydrocarbons comprising at least one carbon to carbon double bond, having the number of carbon atoms designated (i.e., C₂-C₄ alkenyl or C₂₋₄alkenyl means an alkenyl having two to four to eight carbon atoms, C₄-C₈ alkenyl or C₄₋₈alkenyl means an alkenyl having four carbon atoms. Embodiments of alkenyl generally include, but are not limited to, C₂-C₆ alkenyl, such as C₂-C₄ alkenyl, such as C₂-C₃ alkenyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “3-7 membered saturated ring” refers to a mono cyclic non-aromatic saturated radical, wherein each of the atoms forming the ring (i.e., skeletal atoms) is a carbon atom, unless such ring contains one or more heteroatoms if so further defined. 3-7 Membered saturated rings include groups having 3 to 7 ring atoms. Monocyclic 3-7 membered saturated rings include, but are not limited to, cyclopropyl, cyclobutyl, cyclopentyl, cyclohexyl, cycloheptyl.

As used herein, a 3-7 membered saturated ring may optionally contain a heteroatom, said heteroatom being an oxygen, or a nitrogen substituted with H, C₁₋₆alkyl, or C₁₋₆alkoxy-C₁₋₆alkyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two, or three rings), wherein such rings may be attached together in a pendent manner, such as a biphenyl, or may be fused, such as naphthalene. Examples of aryl groups include phenyl, anthracyl, and naphthyl. Preferred examples are phenyl (e.g., C₆-aryl) and biphenyl (e.g., C₁₂-aryl). In some embodiments, aryl groups have from six to sixteen carbon atoms. In some embodiments, aryl groups have from six to twelve carbon atoms (e.g., C₆-C₁₂-aryl). In some embodiments, aryl groups have six carbon atoms (e.g., C₆-aryl).

As used herein, the term “heteroaryl” or “heteroaromatic” refers to a heterocycle having aromatic character. Heteroaryl substituents may be defined by the number of carbon atoms, e.g., C₁-C₉-heteroaryl indicates the number of carbon atoms contained in the heteroaryl group without including the number of heteroatoms. For example, a C₁-C₉-heteroaryl will include an additional one to four heteroatoms. A polycyclic heteroaryl may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Non-limiting examples of polycyclic heterocycles and heteroaryls include indolyl (including, e.g., 3-, 4-, 5-, 6- and 7-indolyl), indolinyl, quinolyl, tetrahydroquinolyl, isoquinolyl (including, e.g., 1- and 5-isoquinolyl), 1,2,3,4-tetrahydroisoquinolyl, cinnolinyl, quinoxalinyl (including, e.g., 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g., 3-, 4-, 5-, 6- and 7-benzofuryl), 2,3-dihydrobenzofuryl, 1,2-benzisoxazolyl, benzothienyl (including, e.g., 3-, 4-, 5-, 6-, and 7-benzothienyl), benzoxazolyl, benzothiazolyl (including, e.g., 2-benzothiazolyl and 5-benzothiazolyl), purinyl, benzimidazolyl (including, e.g., 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group.

As used herein, the terminology “selected from . . . ” (e.g., “R⁴ is selected from A, B and C”) is understood to be equivalent to the terminology “selected from the group consisting of . . . ” (e.g., “R⁴ is selected from the group consisting of A, B and C”).

One embodiment relates to a compound of Formula I as defined herein wherein the carboxylic acid bioisosteres are —S(═O)₂(OH), —P(═O)(OH)₂, —C(═O)NHOH, C(═O)NHCN, 1,2,4-oxadiazol-5 (4H)-one, or 3-hydroxy-4-methylcyclobut-3-ene-1,2-di one. This refers to the following structures:

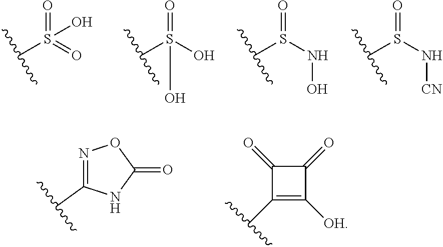

An embodiment relates to a compound of Formula I as defined herein, wherein R⁴ is methyl, or ethyl.

An embodiment relates to a compound of Formula I as defined herein, wherein R⁵ is thiazolyl.

An embodiment relates to a compound of Formula I as defined herein, wherein X is C(═O).

An embodiment relates to a compound of Formula I as defined herein, wherein R⁶ is C₁₋₆alkyl.

An embodiment relates to a compound of Formula I as defined herein, wherein m is 1, n is 0 and r is 1.

An embodiment relates to a compound of Formula I as defined herein, wherein Z is CH₂.

An embodiment relates to a compound of Formula I as defined herein, wherein R⁷ is C₁₋₆alkyl substituted with —COOH, or wherein R⁷ is (CH₂)ₚ-Q-CO₂H.

An embodiment relates to a compound of Formula I as defined herein, wherein Q is phenyl, or wherein Q is a C₃₋₆cycloalkyl, or wherein Q is a 3- to 6-saturated membered ring containing an oxygen.

An embodiment relates to a compound selected from the group consisting of compound satisfying the following formulae:

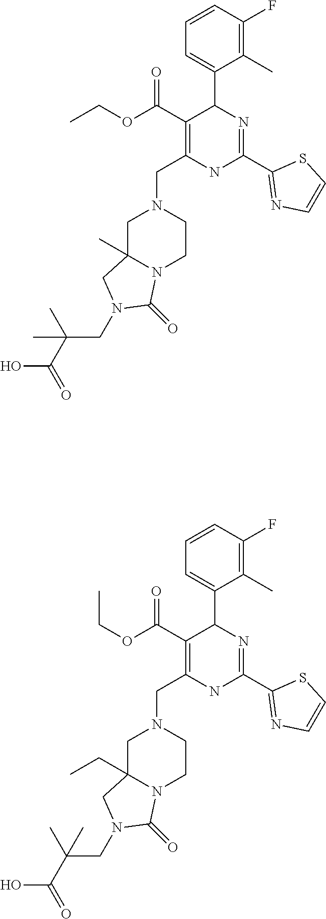 [SMILES: CCOC(=O)C1=C(CN2CCN3C(=O)N(CC(C)(C)C(=O)O)CC3(C)C2)NC(c2nccs2)=NC1c1cccc(F)c1C ; CCOC(=O)C1=C(CN2CCN3C(=O)N(CC(C)(C)C(=O)O)CC3(CC)C2)NC(c2nccs2)=NC1c1cccc(F)c1C]

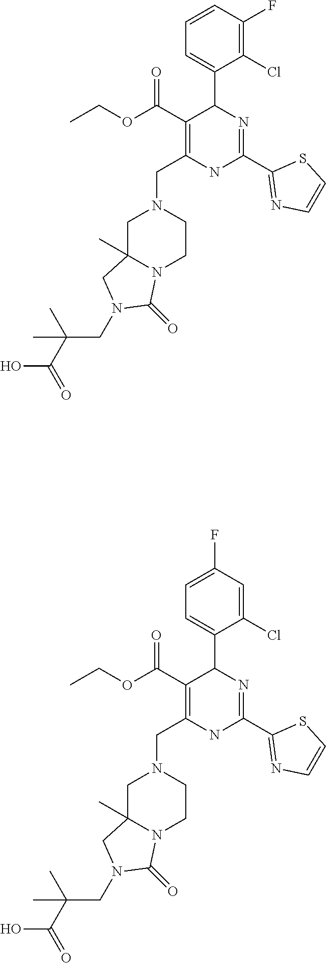 [SMILES: CCOC(=O)C1=C(CN2CCN3C(=O)N(CC(C)(C)C(=O)O)CC3(C)C2)NC(c2nccs2)=NC1c1cccc(F)c1Cl ; CCOC(=O)C1=C(CN2CCN3C(=O)N(CC(C)(C)C(=O)O)CC3(C)C2)NC(c2nccs2)=NC1c1ccc(F)cc1Cl]

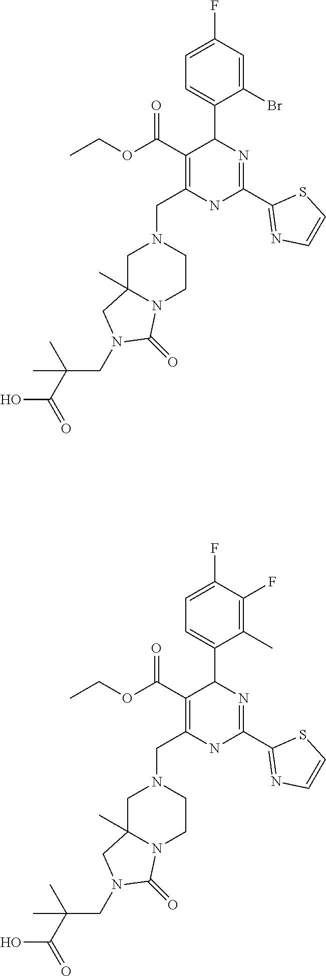 [SMILES: CCOC(=O)C1=C(CN2CCN3C(=O)N(CC(C)(C)C(=O)O)CC3(C)C2)NC(c2nccs2)=NC1c1ccc(F)cc1Br ; CCOC(=O)C1=C(CN2CCN3C(=O)N(CC(C)(C)C(=O)O)CC3(C)C2)NC(c2nccs2)=NC1c1ccc(F)c(F)c1C]

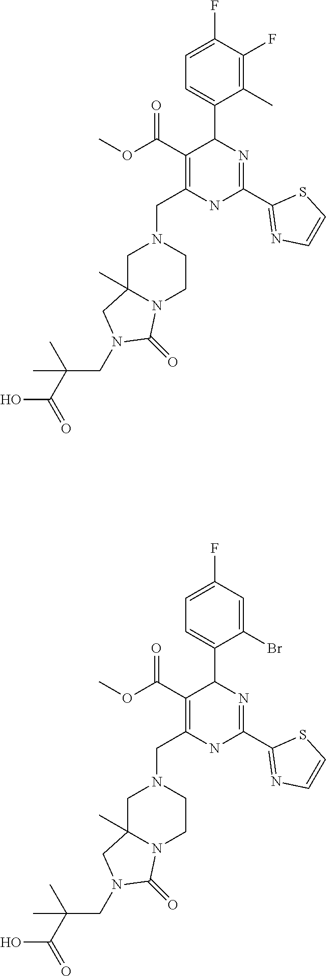 [SMILES: COC(=O)C1=C(CN2CCN3C(=O)N(CC(C)(C)C(=O)O)CC3(C)C2)NC(c2nccs2)=NC1c1ccc(F)c(F)c1C ; COC(=O)C1=C(CN2CCN3C(=O)N(CC(C)(C)C(=O)O)CC3(C)C2)NC(c2nccs2)=NC1c1ccc(F)cc1Br]

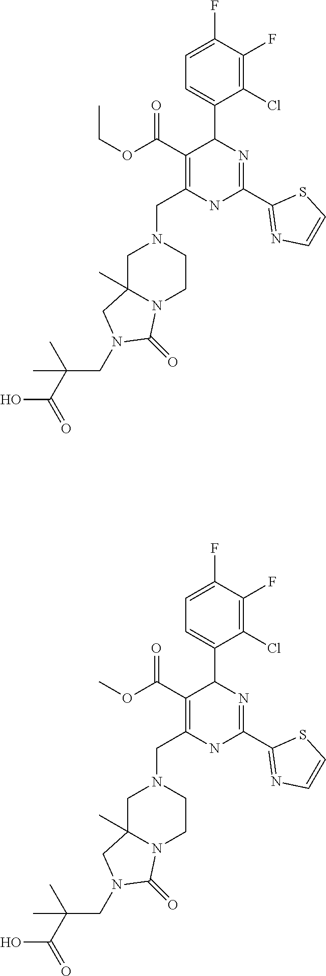 [SMILES: CCOC(=O)C1=C(CN2CCN3C(=O)N(CC(C)(C)C(=O)O)CC3(C)C2)NC(c2nccs2)=NC1c1ccc(F)c(F)c1Cl ; COC(=O)C1=C(CN2CCN3C(=O)N(CC(C)(C)C(=O)O)CC3(C)C2)NC(c2nccs2)=NC1c1ccc(F)c(F)c1Cl]

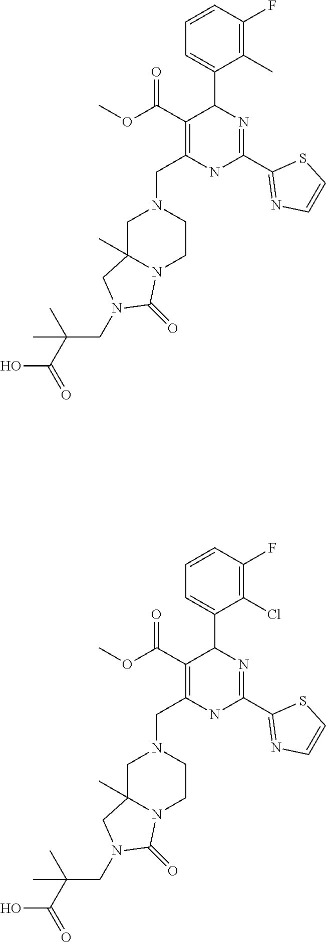 [SMILES: COC(=O)C1=C(CN2CCN3C(=O)N(CC(C)(C)C(=O)O)CC3(C)C2)NC(c2nccs2)=NC1c1cccc(F)c1C ; COC(=O)C1=C(CN2CCN3C(=O)N(CC(C)(C)C(=O)O)CC3(C)C2)NC(c2nccs2)=NC1c1cccc(F)c1Cl]

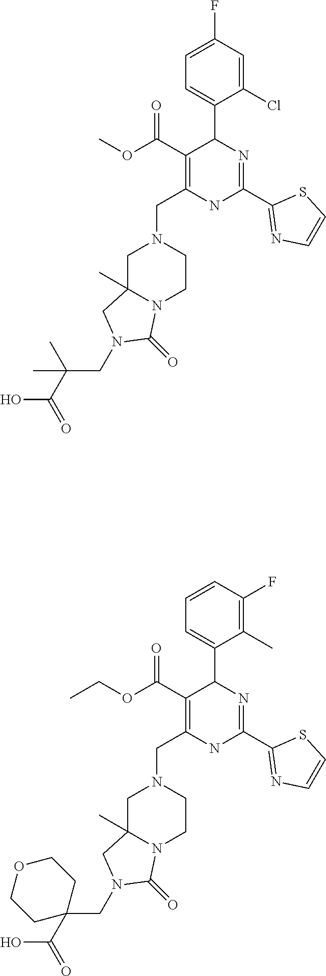 [SMILES: COC(=O)C1=C(CN2CCN3C(=O)N(CC(C)(C)C(=O)O)CC3(C)C2)NC(c2nccs2)=NC1c1ccc(F)cc1Cl ; CCOC(=O)C1=C(CN2CCN3C(=O)N(CC4(C(=O)O)CCOCC4)CC3(C)C2)NC(c2nccs2)=NC1c1cccc(F)c1C]

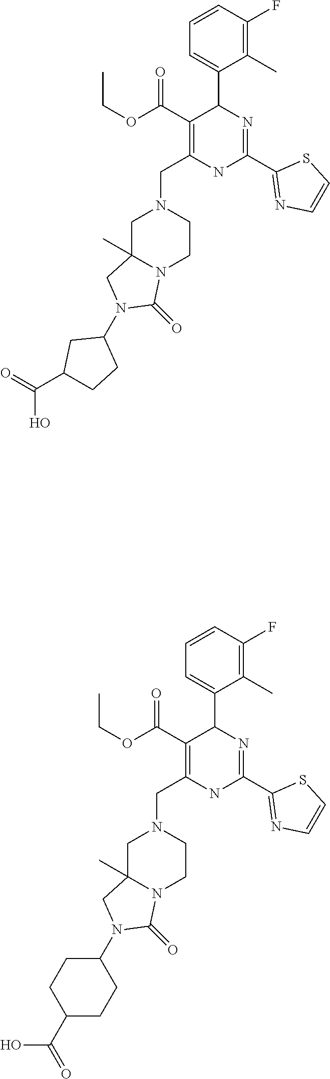 [SMILES: CCOC(=O)C1=C(CN2CCN3C(=O)N(C4CCC(C(=O)O)C4)CC3(C)C2)NC(c2nccs2)=NC1c1cccc(F)c1C ; CCOC(=O)C1=C(CN2CCN3C(=O)N(C4CCC(C(=O)O)CC4)CC3(C)C2)NC(c2nccs2)=NC1c1cccc(F)c1C]

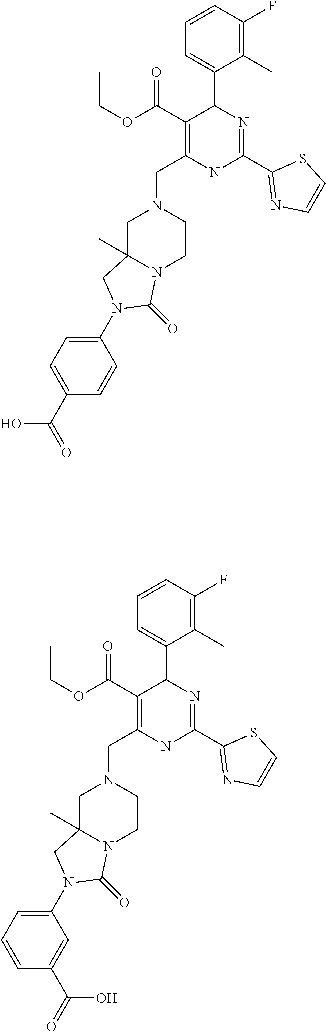 [SMILES: CCOC(=O)C1=C(CN2CCN3C(=O)N(c4ccc(C(=O)O)cc4)CC3(C)C2)NC(c2nccs2)=NC1c1cccc(F)c1C ; CCOC(=O)C1=C(CN2CCN3C(=O)N(c4cccc(C(=O)O)c4)CC3(C)C2)NC(c2nccs2)=NC1c1cccc(F)c1C]

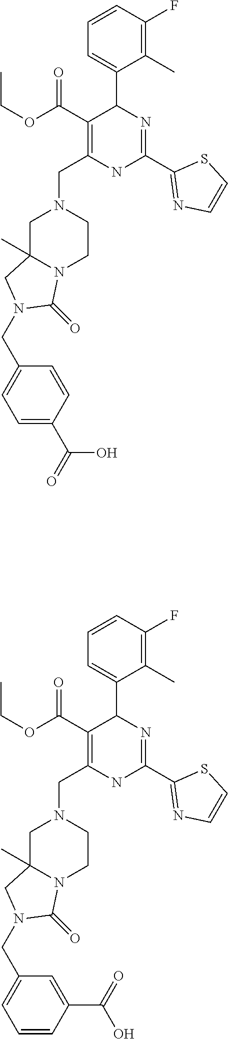 [SMILES: CCOC(=O)C1=C(CN2CCN3C(=O)N(Cc4ccc(C(=O)O)cc4)CC3(C)C2)NC(c2nccs2)=NC1c1cccc(F)c1C ; CCOC(=O)C1=C(CN2CCN3C(=O)N(Cc4cccc(C(=O)O)c4)CC3(C)C2)NC(c2nccs2)=NC1c1cccc(F)c1C]

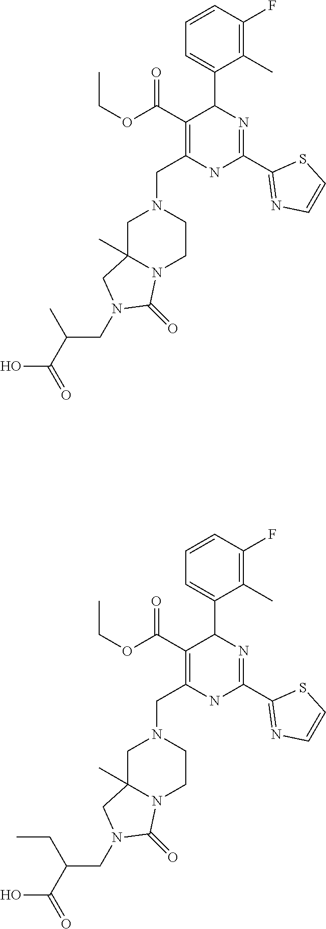 [SMILES: CCOC(=O)C1=C(CN2CCN3C(=O)N(CC(C)C(=O)O)CC3(C)C2)NC(c2nccs2)=NC1c1cccc(F)c1C ; CCOC(=O)C1=C(CN2CCN3C(=O)N(CC(CC)C(=O)O)CC3(C)C2)NC(c2nccs2)=NC1c1cccc(F)c1C]

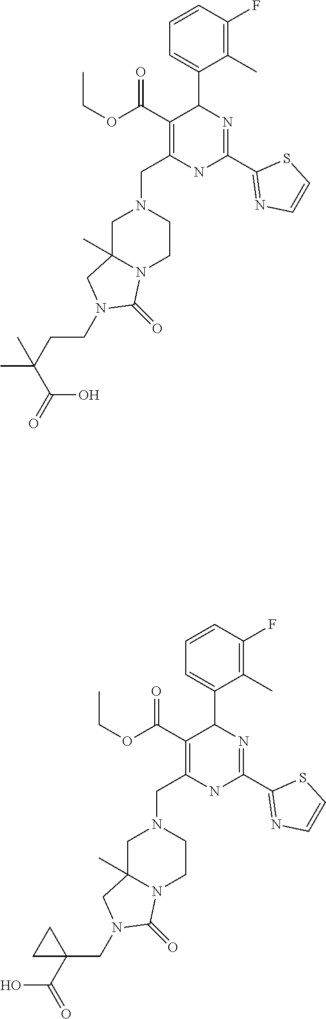 [SMILES: CCOC(=O)C1=C(CN2CCN3C(=O)N(CCC(C)(C)C(=O)O)CC3(C)C2)NC(c2nccs2)=NC1c1cccc(F)c1C ; CCOC(=O)C1=C(CN2CCN3C(=O)N(CC4(C(=O)O)CC4)CC3(C)C2)NC(c2nccs2)=NC1c1cccc(F)c1C]

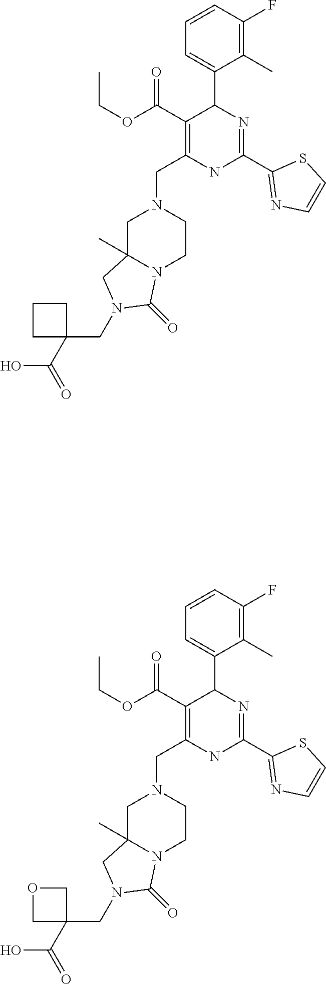 [SMILES: CCOC(=O)C1=C(CN2CCN3C(=O)N(CC4(C(=O)O)CCC4)CC3(C)C2)NC(c2nccs2)=NC1c1cccc(F)c1C ; CCOC(=O)C1=C(CN2CCN3C(=O)N(CC4(C(=O)O)COC4)CC3(C)C2)NC(c2nccs2)=NC1c1cccc(F)c1C]

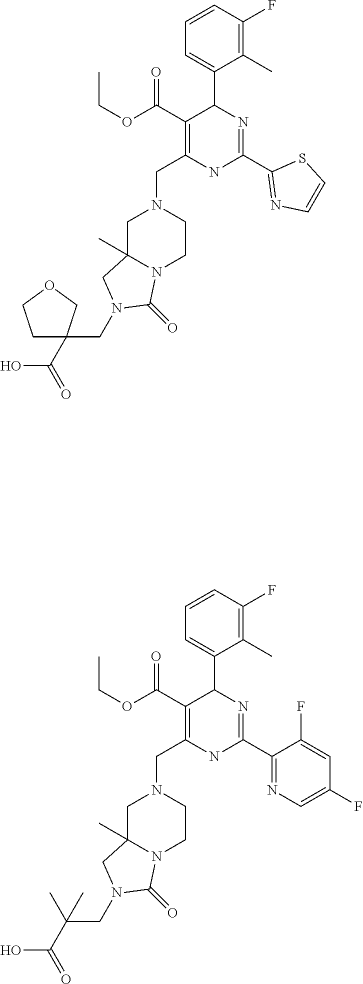 [SMILES: CCOC(=O)C1=C(CN2CCN3C(=O)N(CC4(C(=O)O)CCOC4)CC3(C)C2)NC(c2nccs2)=NC1c1cccc(F)c1C ; CCOC(=O)C1=C(CN2CCN3C(=O)N(CC(C)(C)C(=O)O)CC3(C)C2)NC(c2ncc(F)cc2F)=NC1c1cccc(F)c1C]

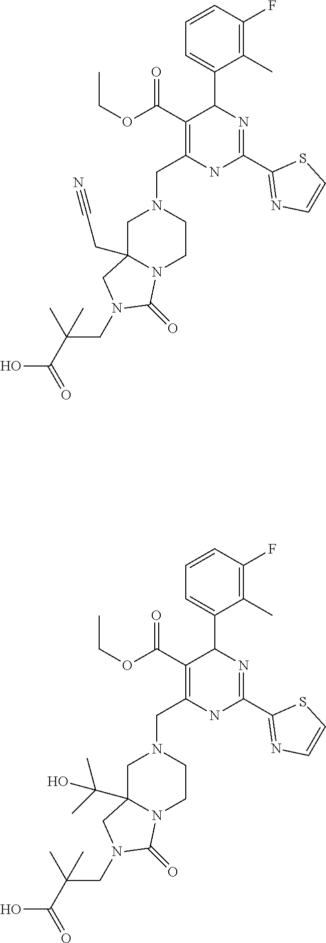 [SMILES: CCOC(=O)C1=C(CN2CCN3C(=O)N(CC(C)(C)C(=O)O)CC3(CC#N)C2)NC(c2nccs2)=NC1c1cccc(F)c1C ; CCOC(=O)C1=C(CN2CCN3C(=O)N(CC(C)(C)C(=O)O)CC3(C(C)(C)O)C2)NC(c2nccs2)=NC1c1cccc(F)c1C]

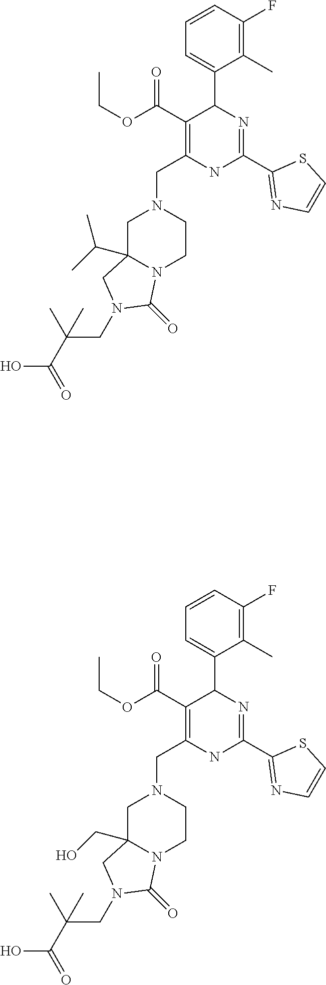 [SMILES: CCOC(=O)C1=C(CN2CCN3C(=O)N(CC(C)(C)C(=O)O)CC3(C(C)C)C2)NC(c2nccs2)=NC1c1cccc(F)c1C ; CCOC(=O)C1=C(CN2CCN3C(=O)N(CC(C)(C)C(=O)O)CC3(CO)C2)NC(c2nccs2)=NC1c1cccc(F)c1C]

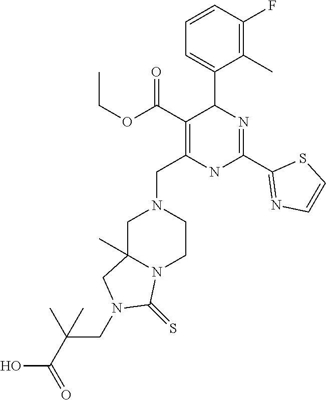 [SMILES: CCOC(=O)C1=C(CN2CCN3C(=S)N(CC(C)(C)C(=O)O)CC3(C)C2)NC(c2nccs2)=NC1c1cccc(F)c1C]

An embodiment relates to a compound selected from the group consisting of compound satisfying the following formulae:

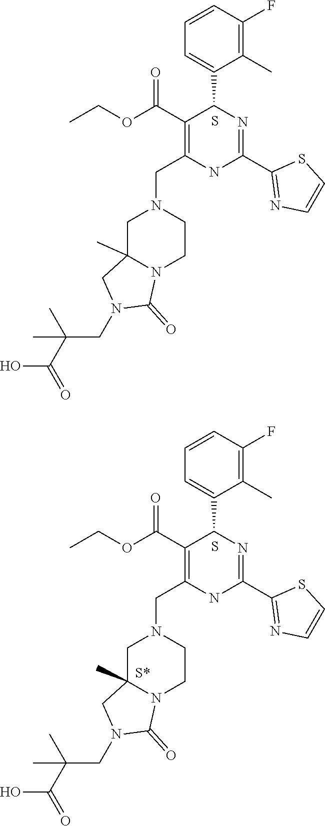

An embodiment relates to a compound selected from the group consisting of compound satisfying the following formulae:

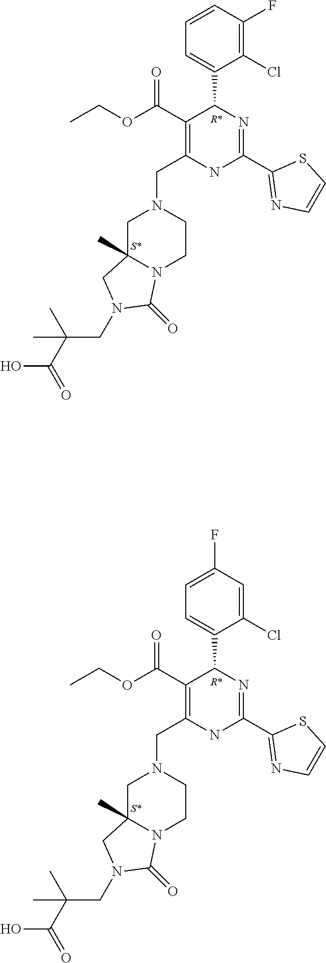 [SMILES: CCOC(=O)C1=C(CN2CCN3C(=O)N(CC(C)(C)C(=O)O)C[C@]3(C)C2)NC(c2nccs2)=N[C@H]1c1cccc(F)c1Cl ; CCOC(=O)C1=C(CN2CCN3C(=O)N(CC(C)(C)C(=O)O)C[C@]3(C)C2)NC(c2nccs2)=N[C@H]1c1ccc(F)cc1Cl]

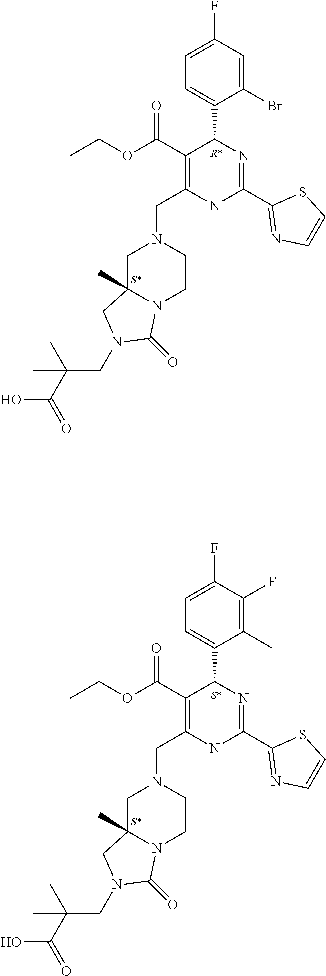 [SMILES: CCOC(=O)C1=C(CN2CCN3C(=O)N(CC(C)(C)C(=O)O)C[C@]3(C)C2)NC(c2nccs2)=N[C@H]1c1ccc(F)cc1Br ; CCOC(=O)C1=C(CN2CCN3C(=O)N(CC(C)(C)C(=O)O)C[C@]3(C)C2)NC(c2nccs2)=N[C@H]1c1ccc(F)c(F)c1C]

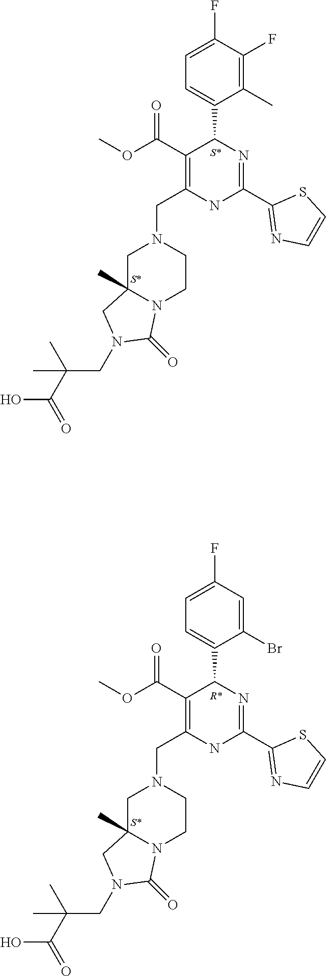 [SMILES: COC(=O)C1=C(CN2CCN3C(=O)N(CC(C)(C)C(=O)O)C[C@]3(C)C2)NC(c2nccs2)=N[C@H]1c1ccc(F)c(F)c1C ; COC(=O)C1=C(CN2CCN3C(=O)N(CC(C)(C)C(=O)O)C[C@]3(C)C2)NC(c2nccs2)=N[C@H]1c1ccc(F)cc1Br]

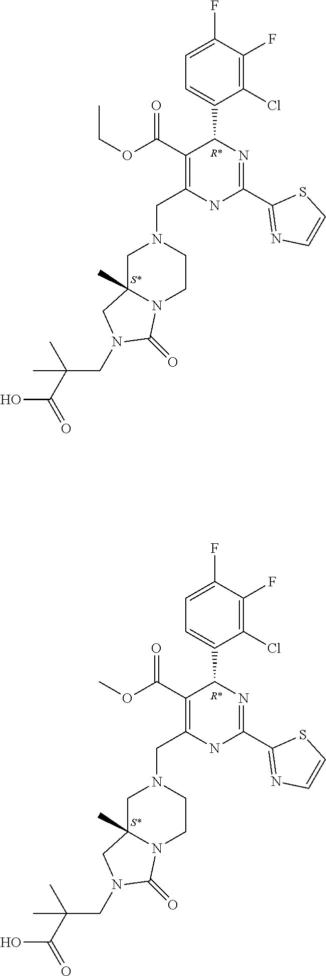 [SMILES: CCOC(=O)C1=C(CN2CCN3C(=O)N(CC(C)(C)C(=O)O)C[C@]3(C)C2)NC(c2nccs2)=N[C@H]1c1ccc(F)c(F)c1Cl ; COC(=O)C1=C(CN2CCN3C(=O)N(CC(C)(C)C(=O)O)C[C@]3(C)C2)NC(c2nccs2)=N[C@H]1c1ccc(F)c(F)c1Cl]

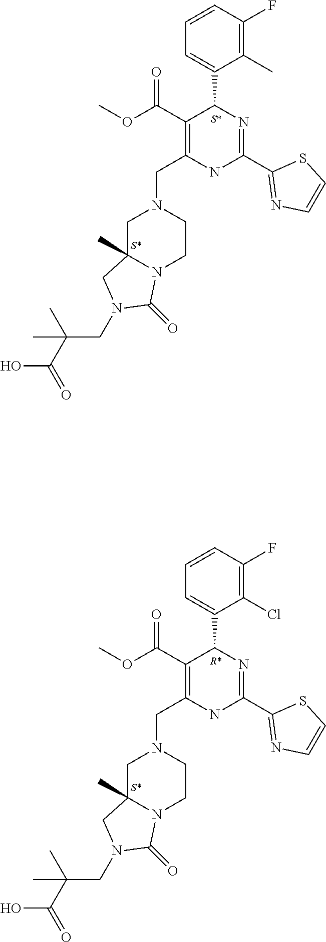 [SMILES: COC(=O)C1=C(CN2CCN3C(=O)N(CC(C)(C)C(=O)O)C[C@]3(C)C2)NC(c2nccs2)=N[C@H]1c1cccc(F)c1C ; COC(=O)C1=C(CN2CCN3C(=O)N(CC(C)(C)C(=O)O)C[C@]3(C)C2)NC(c2nccs2)=N[C@H]1c1cccc(F)c1Cl]

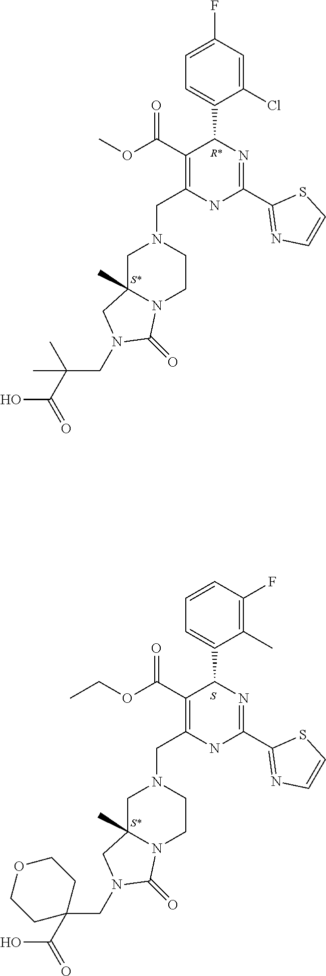

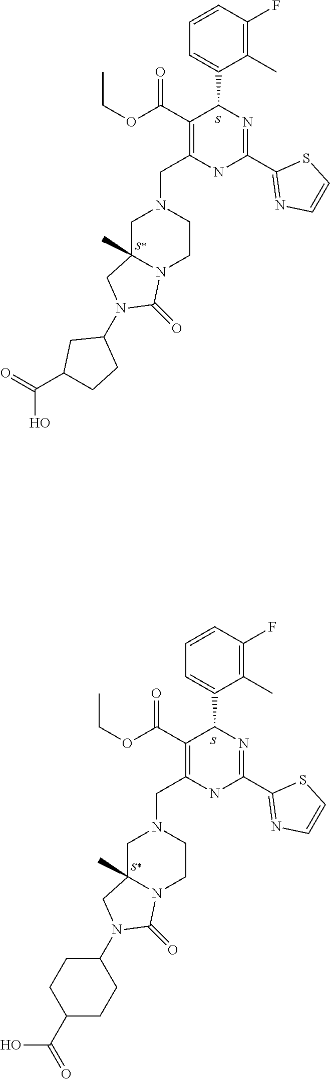

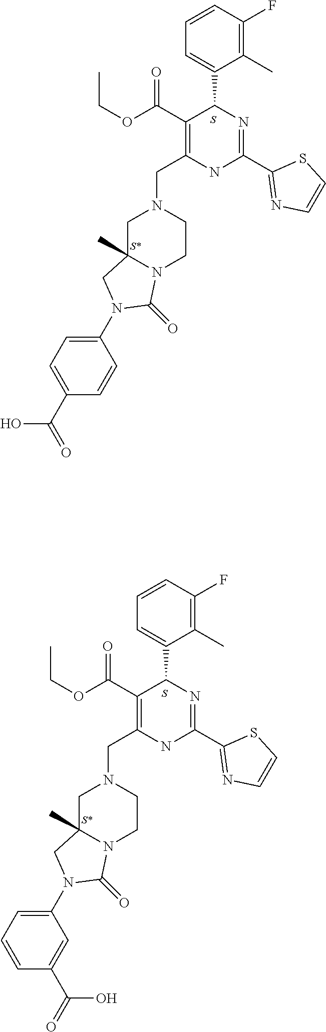

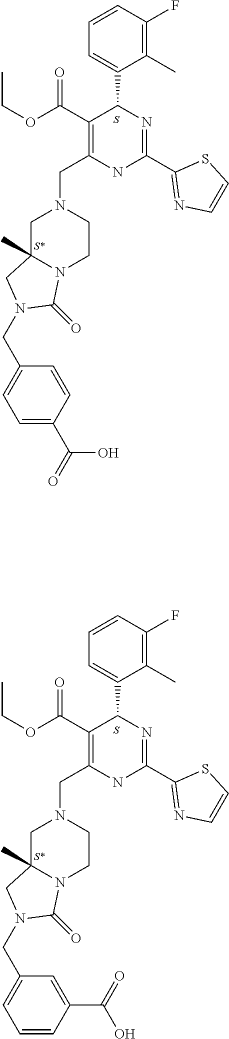

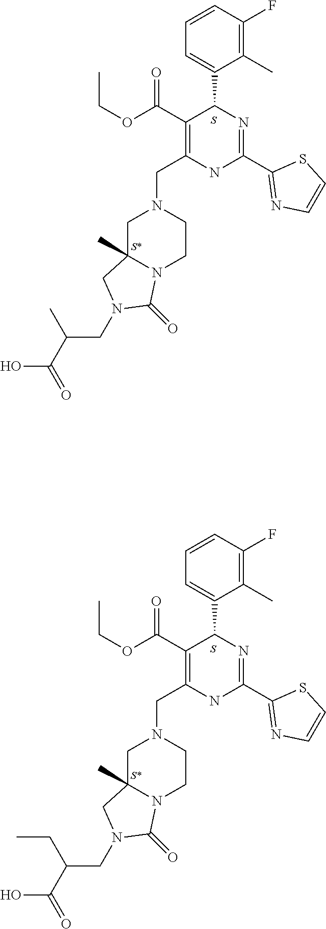

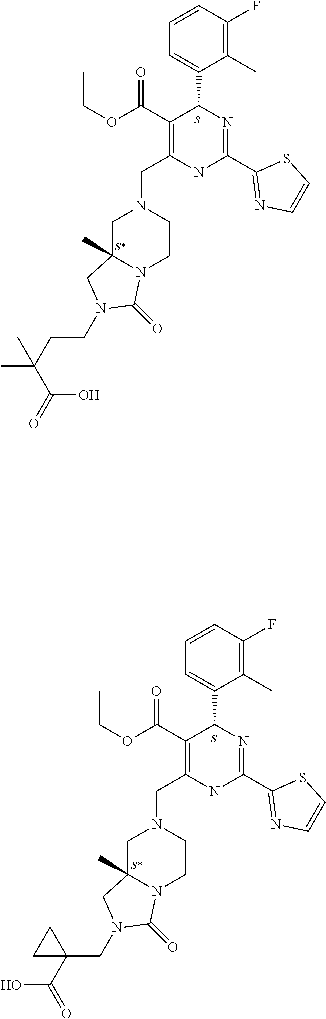

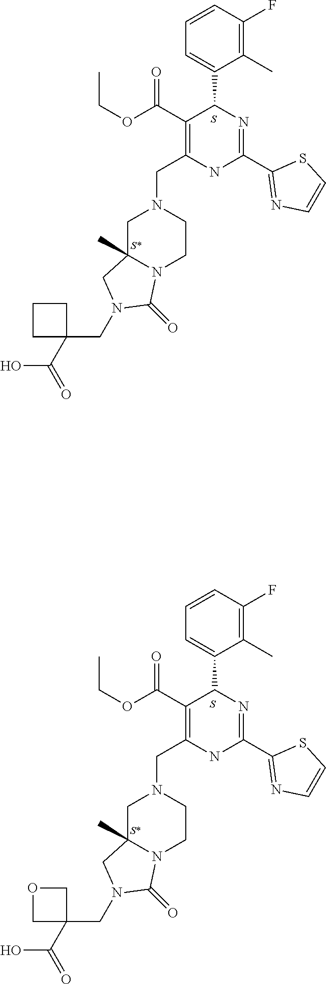

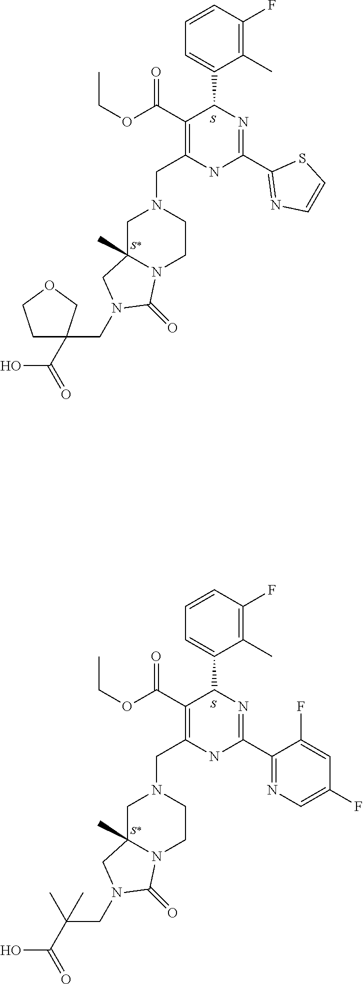

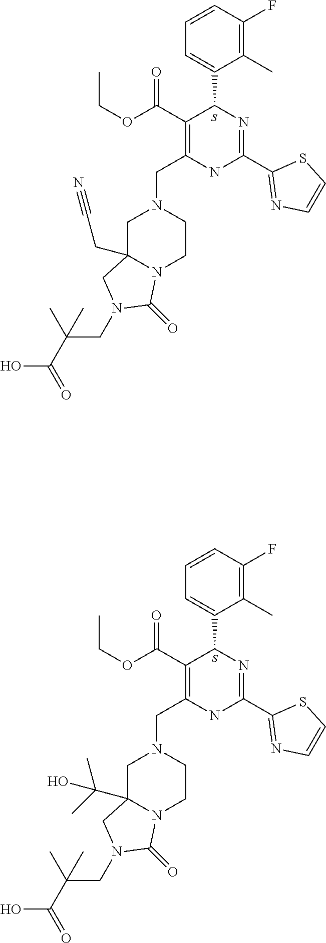

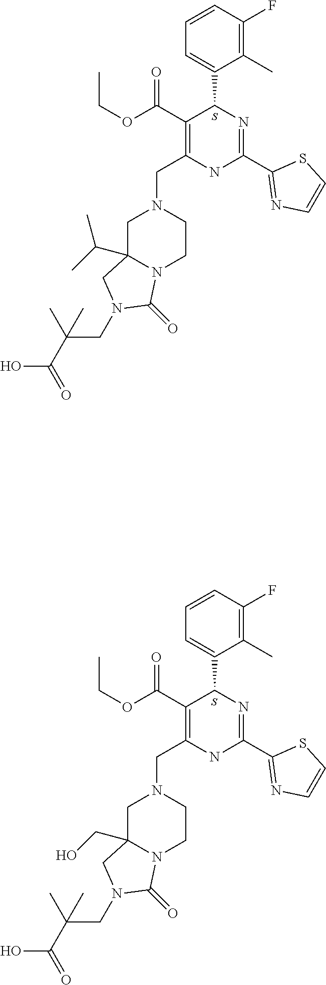

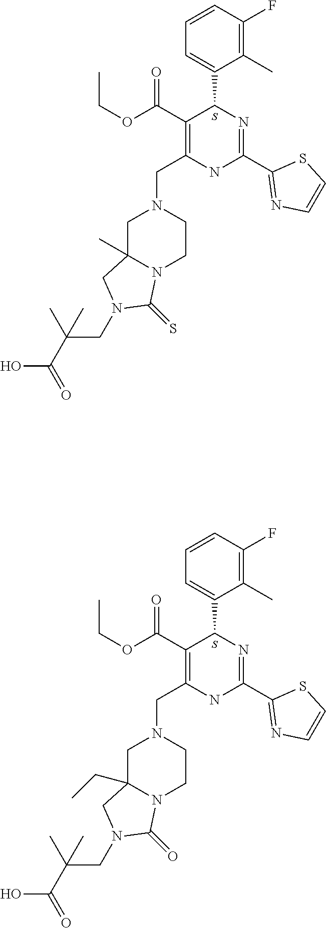

The disclosed compounds may possess one or more stereocenters, and each stereocenter may exist independently in either the R or S configuration. For some compounds, the stereochemical configuration at indicated centres has been assigned as “R*”, “S*”, “*R” or (*S) when the absolute stereochemistry is undetermined although the compound itself has been isolated as a single stereoisomer and is enantiomerically/diastereomerically pure. In an embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the disclosed compound described herein. In another embodiment, compounds described herein contain one or more chiral centers. These compounds are prepared by any means, including stereoselective synthesis, enantioselective synthesis or separation of a mixture of enantiomers or diastereomers. Resolution of compounds and isomers thereof is achieved by any means including, by way of non-limiting example, chemical processes, enzymatic processes, fractional crystallization, distillation, and chromatography.

When the absolute R or S stereochemistry of a compound cannot be determined, it can be identified by the retention time after chromatography under particular chromatographic conditions as determined by chromatography column, eluent etc.

In one embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In one embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In an embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and techniques known to a person skilled in the art. General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources or are prepared using procedures described herein.

Compounds of the application also includes intermediate compounds, including any salts thereof, such as

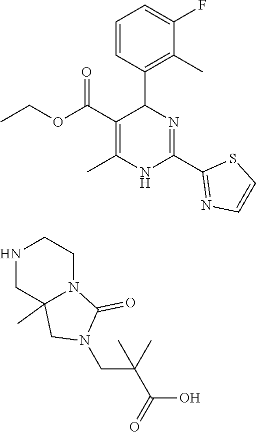 [SMILES: CCOC(=O)C1=C(C)NC(c2nccs2)=NC1c1cccc(F)c1C ; CC(C)(CN1CC2(C)CNCCN2C1=O)C(=O)O]

### Methods

Provided herein is a method of treating an HBV infection in an individual in need thereof, comprising administering to the individual a therapeutically effective amount of a disclosed compound.

Also provided herein is a method of eradicating an HBV infection in an individual in need thereof, comprising administering to the individual a therapeutically effective amount of a disclosed compound.

Provided herein is a method of reducing viral load associated with an HBV infection in an individual in need thereof, comprising administering to the individual a therapeutically effective amount of a disclosed compound.

Further, provided herein is a method of reducing reoccurrence of an HBV infection in an individual in need thereof, comprising administering to the individual a therapeutically effective amount of a disclosed compound.

Provided herein is a method of inhibiting or reducing the formation or presence of HBV DNA-containing particles or HBV RNA-containing particles in an individual in need thereof, comprising administering to the individual a therapeutically effective amount of a disclosed compound.

In certain aspects, the methods and/or compositions described herein are effective for inhibiting or reducing the formation or presence of HBV-associated particles in vitro or in vivo (e.g., in a cell, in a tissue, in an organ (e.g., in the liver), in an organism or the like). HBV-associated particles may contain HBV DNA (i.e., linear and/or covalently closed circular DNA (cccDNA)) and/or HBV RNA (i.e., pre-genomic RNA and/or sub-genomic RNA). Accordingly, HBV-associated particles include HBV DNA-containing particles or HBV RNA-containing particles.

As used herein, “HPV-associated particles” refer to both infectious HBV virions (i.e., Dane particles) and non-infectious HBV subviral particles (i.e., HBV filaments and/or HBV spheres). HBV virions comprise an outer envelope including surface proteins, a nucleocapsid comprising core proteins, at least one polymerase protein, and an HBV genome. HBV filaments and HBV spheres comprise HBV surface proteins, but lack core proteins, polymerase and an HBV genome. HBV filaments and HBV spheres are also known collectively as surface antigen (HBsAg) particles. HBV spheres comprise middle and small HBV surface proteins. HBV filaments also include middle, small and large HBV surface proteins.

HBV subviral particles can include the nonparticulate or secretory HBeAg, which serves as a marker for active replication of HBV.

Provided herein is a method of reducing an adverse physiological impact of an HBV infection in an individual in need thereof, comprising administering to the individual a therapeutically effective amount of a disclosed compound.

Also provided herein is a method of reducing, slowing, or inhibiting an HBV infection in an individual in need thereof, comprising administering to the individual a therapeutically effective amount of a disclosed compound.

Provided herein is a method of inducing reversal of hepatic injury from an HBV infection in an individual in need thereof, comprising administering to the individual a therapeutically effective amount of a disclosed compound.

Provided herein is a method of reducing the physiological impact of long-term antiviral therapy for HBV infection in an individual in need thereof, comprising administering to the individual a therapeutically effective amount of a disclosed compound.

Provided herein is a method of prophylactically treating an HBV infection in an individual in need thereof, wherein the individual is afflicted with a latent HBV infection, comprising administering to the individual a therapeutically effective amount of a disclosed compound.

In an embodiment, the individual is refractory to other therapeutic classes of HBV drugs (e.g., HBV polymerase inhibitors, interferons, viral entry inhibitors, viral maturation inhibitors, literature-described capsid assembly modulators, antiviral compounds of distinct or unknown mechanism, and the like, or combinations thereof). In another embodiment, the disclosed method reduces viral load in an individual suffering from an HBV infection to a greater extent or at a faster rate compared to the extent that other therapeutic classes of HBV drugs reduce viral load in the individual.

In an embodiment, the administering of a disclosed compound, or a pharmaceutically acceptable salt thereof, allows for administering of the at least one additional therapeutic agent at a lower dose or frequency as compared to the administering of the at least one additional therapeutic agent alone that is required to achieve similar results in prophylactically treating an HBV infection in an individual in need thereof.

In an embodiment, the administering of a disclosed compound, or a pharmaceutically acceptable salt thereof, reduces the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In an embodiment, the disclosed method reduces viral load in an individual suffering from an HBV infection, thus allowing lower doses or varying regimens of combination therapies to be used.

In an embodiment, the disclosed method causes a lower incidence of viral mutation or viral resistance compared to other classes of HBV drugs, thereby allowing for long term therapy and minimizing the need for changes in treatment regimens.

In an embodiment, the administering of a compound the invention, or a pharmaceutically acceptable salt thereof, causes a lower incidence of viral mutation or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In an embodiment, the disclosed method increases the seroconversion rate from HBV infected to non-HBV infected or from detectable HBV viral load to non-detectable HBV viral load beyond that of current treatment regimens. As used herein, “seroconversion” refers to the period of time during which HBV antibodies develop and become detectable.

In an embodiment, the disclosed method increases or normalizes or restores normal health, elicits full recovery of normal health, restores life expectancy, or resolves the viral infection in the individual in need thereof.

In an embodiment, the disclosed method eliminates or decreases the number of HBV RNA particles that are released from HBV infected cells thus enhancing, prolonging, or increasing the therapeutic benefit of the disclosed compounds.

In an embodiment, the disclosed method eradicates HBV from an individual infected with HBV, thereby obviating the need for long term or life-long treatment, or shortening the duration of treatment, or allowing for reduction in dosing of other antiviral agents.

In another embodiment, the disclosed method further comprises monitoring or detecting the HBV viral load of the subject, and wherein the method is carried out for a period of time including until such time that the HBV virus is undetectable.

Accordingly, in an embodiment, provided herein is a method of treating an HBV infection in an individual in need thereof, comprising administering to the individual a therapeutically effective amount of a compound of Formula I, or a pharmaceutically acceptable salt thereof.

Accordingly, in an embodiment, provided herein is a method of treating an HBV infection in an individual in need thereof, comprising administering to the individual a therapeutically effective amount of a compound of Formula I, or a pharmaceutically acceptable salt thereof.

In another embodiment, provided herein is a method of treating an HBV infection in an individual in need thereof, comprising administering to the individual a therapeutically effective amount of a compound of Table 1, or a pharmaceutically acceptable salt thereof.

In an embodiment of any of the methods provided herein, the method can further comprise monitoring the HBV viral load of the subject, wherein the method is carried out for a period of time such that the HBV virus is undetectable.

### Combination Therapies

The disclosed compounds may be useful in combination with one or more additional compounds useful for treating HBV infection, or a HBV-associated or -induced disease, or a liver disease. These additional compounds may comprise other disclosed compounds and/or compounds known to treat, prevent, or reduce the symptoms or effects of HBV infection, or of an HBV-associated or -induced disease, or of a liver disease.

Particularly, in an aspect a product is provided comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt of the application or the pharmaceutical composition of the application, and wherein said second compound is another HBV inhibitor which is selected from the group consisting of HBV combination drugs, HBV DNA polymerase inhibitors, immunomodulators toll-like (TLR) receptor modulators, interferon alpha receptor ligands, hyaluronidase inhibitors, hepatitis b surface antigen (HbsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, farnesoid X receptor agonists, HBV antibodies, CCR2 chemokine antagonists, thymosin agonists, cytokines, nucleoprotein modulators, retinoic acid-inducible gene 1 stimulators, NOD2 stimulators, phosphatidylinositol 3-kinase (P13K) inhibitors, indoleamine 2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and (other) anti-HBV drugs.

The one or more additional compounds may e.g., be selected from interferon (for example, interferon-alpha-2a is pegylated interferon-alpha-2a (PEGASYS)), nucleoside or nucleotide or non-nucleos(t)ide polymerase inhibitors, immunomodulatory agents (e.g., IL-12, IL-18, IFN-alpha, -beta, and -gamma and TNF-alpha among others), TLR agonists, siRNAs and antisense oligonucleotides.

In another embodiment, the disclosed compound and the at least one additional therapeutic agent are co-formulated. In yet another embodiment, the disclosed compound and the at least one additional therapeutic agent are co-administered.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In an embodiment of any of the methods of administering combination therapies provided herein, the method can further comprise monitoring or detecting the HBV viral load of the subject, wherein the method is carried out for a period of time including until such time that the HBV virus is undetectable.

### Administration/Dosage/Formulations

In another aspect, provided herein is a pharmaceutical composition comprising at least one disclosed compound, or a pharmaceutically acceptable salt thereof, together with a pharmaceutically acceptable carrier.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

In particular embodiments, the compound is formulated in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of HBV infection in a patient.

In an embodiment, the compositions of the invention are formulated using one or more pharmaceutically acceptable excipients or carriers. In an embodiment, the pharmaceutical compositions of the invention comprise a therapeutically effective amount of a disclosed compound and a pharmaceutically acceptable carrier.

In some embodiments, the dose of a disclosed compound is from about 1 mg to about 2,500 mg. In some embodiments, a dose of a disclosed compound used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In an embodiment, the present invention is directed to a packaged pharmaceutical composition comprising a container holding a therapeutically effective amount of a disclosed compound, alone or in combination with a second pharmaceutical agent; and instructions for using the compound to treat, prevent, or reduce one or more symptoms of HBV infection in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be un-coated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the invention.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### EXAMPLES

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

### Preparative Examples

Unless otherwise indicated, LCMS and NMR was conducted by using one of the following general methods.

General Methods of LCMS and NMR

General Procedure A

The LCMS measurement was performed using an Agilent system comprising a binary pump with degasser, an autosampler, a column oven (set at 40° C., unless otherwise indicated) and a column as specified in the respective methods below. Flow from the column was split to a MS and UV spectrometer. The MS detector was configured with an electrospray ionization source. Mass spectra were acquired by scanning from 100 to 1000 in 1.06 sec/cycle. The capillary voltage was 3 kV for positive ionization mode and 2.5 kV for negative ionization mode and the source temperature was maintained at 100° C. Nitrogen was used as the nebulizer gas. Data acquisition was performed with an Agilent ChemStation data system.

Method 1

In addition to the general procedure A: reversed phase LCMS for quality control was performed by Agilent 1200 with a diode-array detector (DAD) and carried out on a Sunfire C18 column (5 μm, 4.6×50 mm) with a flow rate of 1.5 ml/min. Two mobile phases (mobile phase A1: 0.02% ammonium acetate in water; mobile phase A2: 0.1% TFA in water; mobile phase B1: acetonitrile) were employed to run a gradient condition from 95% A1 or A2 and 5% B to 5% A1 or A2 and 95% B in 4.0 minutes. An injection volume of 1˜10 μl was used.

Method 2

In addition to the general procedure A: reversed phase LCMS for monitoring the reactions was performed by Agilent 1260 with a variable wavelength detector (VWD) and carried out on a Dikma Diamonsil plus C18 column (5 μm, 4.6×30 mm) with a flow rate of 2.0 ml/min. Two mobile phases (mobile phase A1: H₂O+0.02% ammoniumacetate+5% ACN; mobile phase A2: H₂O+0.1% TFA+5% ACN; mobile phase B: acetonitrile) were employed to run a gradient condition from 95% A1 or A2 and 5% B to 5% A1 or A2 and 95% B in 1.4 minutes. An injection volume of 1-5 μl was used.

Method 3

In addition to the general procedure A: reversed phase LCMS for monitoring the reactions was performed by Agilent 6120 (stationary phase Sunfire C18 2.5 μm, 3.0×30 mm. Mobile phase: 0.01% FA solution in water, and ACN, Gradient from 5% ACN to 95% in 2.5 min and stay in 95% for 1 min.

General Procedure B

The LCMS measurement was performed using a UPLC (Ultra Performance Liquid Chromatography) Acquity (Waters) system comprising a quaternary pump with degasser, an autosampler, a photo-diode array detector (PDA) and a column as specified in the respective methods below, the column is hold at a temperature of 40° C. Flow from the column was brought to MS detector. The MS detector was configured with an electrospray ionization source. Mass spectra were acquired by scanning from 100 to 1000 in 0.25 sec/cycle. The capillary needle voltage was 3 kV and the source temperature was maintained at 120° C. Cone voltage was 30 V for positive ionization mode and 30 V for negative ionization mode. Nitrogen was used as the nebulizer gas. Data acquisition was performed with a Waters-Micromass MassLynx-Openlynx data system.

Reversed phase UPLC was carried out on a Waters Acquity BEH (bridged ethylsiloxane/silica hybrid) C18 column (1.7 μm, 2.1×50 mm) with a flow rate of 0.5 ml/min. Two mobile phases (mobile phase A: 95% (H₂O+0.02% ammoniumacetate+5% ACN); mobile phase B: acetonitrile; mobile phase C: 95% (H₂O+0.1% TFA+5% ACN) were employed to run a gradient condition from 95% A or C and 5% B to 5% A or C and 95% B in 1 minute. An injection volume of 0.5 μl was used.

General Procedure C

The reversed phase preparation was performed using a system comprising two unit pumps without degasser, a UV/Vis detector and a column as specified in the respective methods below. Flow from the column was split to a UV spectrometer.

Method 1

In addition to the general procedure C: Prep-reversed phase LC was carried out on a Gilson with an autosampler, an Xbridge prep C18 OBD column (5 μm, 19×150 mm) with a flow rate of 15-20 ml/min. Two mobile phases (mobile phase A1: H₂O (0.1% Ammonium bicarbonate); mobile phase A2: H₂O (Ammonium hydroxide); mobile phase A3: H₂O (0.1% TFA); mobile phase B: acetonitrile) were employed to run a gradient condition from 95% A1 or A2 or A3 and 5% B to 20% A1 or A2 or A3 and 80% B. Data acquisition was performed with a Trilution LC data system.

Method 2

In addition to the general procedure C: reversed phase preparation was carried out on a automatic medium pressure flash separation—Compact Purifier from Lisure Science Ltd. with reversed phase SW-5231 C18 column (40-60 μm, 120□, 18 g, 40 g, 130 g) with a flow rate of 30-100 ml/min. Two mobile phases (mobile phase A1: H₂O (0.1% Ammonium bicarbonate); mobile phase A2: H₂O (Ammonium hydroxide); mobile phase A3: H₂O (0.1% Hydrochloric acid); mobile phase A4: H₂O; mobile phase B: acetonitrile) were employed to run a gradient condition from 95% A1 or A2 or A3 or A4 and 5% B to 5% A1 or A2 or A3 or A4 and 95% B. Data acquisition was performed with a Compact data system.

Method 3

In addition to the general procedure C: Prep-reversed phase LC was carried out on a Waters with an autosampler, a Xbridge prep C18 OBD column (Sum, 19*150 mm) with a flow rate of 20 ml/min. Two mobile phases (mobile phase A: H₂O (0.1% Ammonium bicarbonate); mobile phase B: acetonitrile) were employed to run a gradient condition from 95% A and 5% B to 50% A and 50% B. Data acquisition was performed with a Waters MassLynx data system.

General Procedure D

The chiral measurement was performed using a system comprising an autosampler, a column oven (set at ambient, unless otherwise indicated), a diode-array detector (DAD) and a column as specified in the respective methods below. Flow from the column was split to a UV spectrometer. LC spectra were acquired by scanning from 190 nm to 400 nm with deuterium lamp and from 401 nm to 800 nm with tungsten lamp using a slit width of 1.2 nm. The chiral chiralpak or chiralcel columns from Daicel Chiral technologies (China) Ltd. are divided into two types according to the different stuffings: Type 1: IA, IB, IC, ID, IE, IF, IG, IH; Type 2: AD-H, AS-H, OD-H, OJ-H.

Method 1:

In addition to the general procedure D: Chiral HPLC was carried out on an Agilent 1200 or Shimadzu LC-20A with a quaternary pump with degasser, a chiral column (Sum, 4.6*250 mm) with a flow rate of 1.0 ml/min for chiral analysis or a chiral column (Sum, 20*250 mm) with a flow rate of 10-20 ml/min for chiral preparation. The mobile phases are the different ratios among MeOH, EtOH, Hex, IPA etc. Data acquisition was performed with an Agilent ChemStation or Shimadzu Lab Solutions data system.

Method 2:

In addition to the general procedure D: chiral analysis was carried out on a Waters-TharSFC with a column oven (40° C.) with a flow rate of 2-3 ml/min and data acquisition was performed with TharSFC Chrom Scope data system. Chiral-preparation was carried out on a Waters-SFC-80 with a flow rate of 45-60 ml/min and data acquisition was performed with Waters-TharSFC SuperChrom data system. The mobile phase is CO₂ and MeOH, EtOH can be used as co-solvents.

General Procedure E

The below NMR experiments were carried out using a NMR spectrometers at ambient temperature, using internal deuterium lock and equipped with BBO 400 MHz S1 5 mm with Z-gradient; PLUS (²H, ¹H, BBF) probe head for the 400 MHz and DUL 300 MHz S1 5 mm Z-gradient (²H, ¹H, ¹³C) probe head for the 300 MHz. Chemical shifts (δ) are reported in parts per million (ppm).

Method 1:

In addition to the general procedure E: A Bruker Avance III 400 MHz spectrometer was used to measure the NMR experiment.

Method 2:

In addition to the general procedure E: A Bruker Avance Neo 400 MHz spectrometer was used to measure the NMR experiment.

Method 3:

In addition to the general procedure E: A ZKNJ BIXI-1 300 MHz spectrometer was used to measure the NMR experiment.

Method 4:

In addition to the general procedure E: A Bruker Ascend 400 MHz spectrometer was used to measure the NMR experiment.

Exemplary compounds useful in methods of the invention will now be described by reference to the illustrative synthetic schemes for their general preparation below and the specific examples to follow.

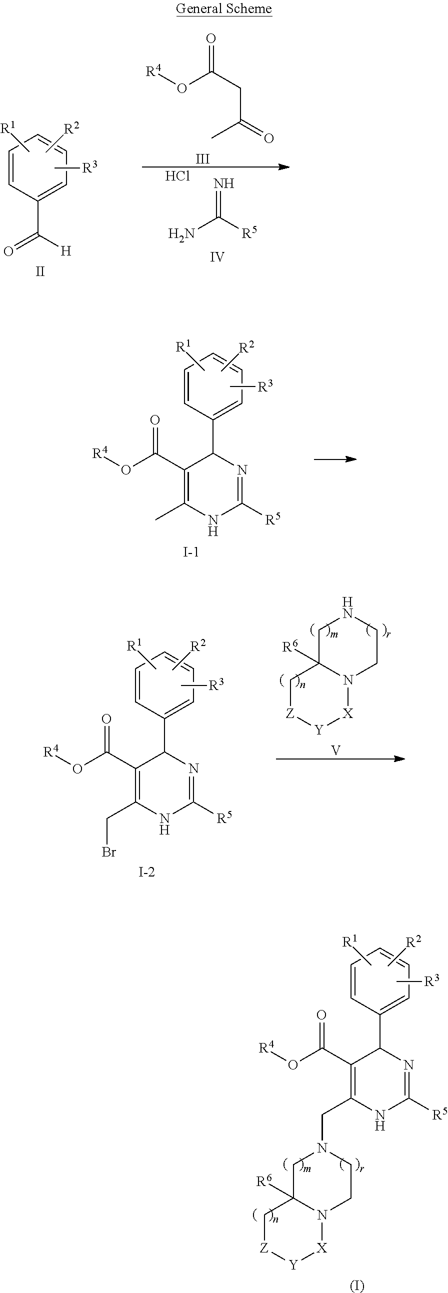

The general synthesis of compound of general formula I is described in general scheme. Intermediate I-1 can be prepared from the condensation of aldehyde II, acetoacetate III and amidine IV in the presence of a base such as NaOAc. Racemic compound I can be separated by SFC to give two enantiomers. Compound I-1 was converted to compound 1-2 using brominating reagent such as N-Bromosuccinimide. Coupling of compound 1-2 and compound V in the presence of a base, such as triethanolamine, produces compound I.

### Intermediate H1: Ethyl 4-(2-chloro-3-fluorophenyl)-6-methyl-2-(thiazol-2-yl)-1,4-dihydropyrimidine-5-carboxylate

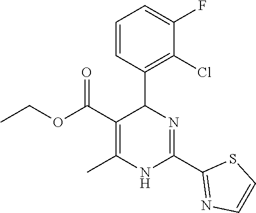 [SMILES: CCOC(=O)C1=C(C)NC(c2nccs2)=NC1c1cccc(F)c1Cl]

To a solution of 2-chloro-3-fluorobenzaldehyde (8.8 g, 55.7 mmol), ethyl 3-oxobutanoate (7.24 g, 55.7 mmol) in isopropanol (40 mL) was added piperidine (473 mg, 5.57 mmol) and AcOH (334 mg, 5.57 mmol). After stirred at room temperature for 4 hours, the mixture was added thiazole-2-carboximidamide (6.4 g, 39 mmol) and triethylamine (5.62 g, 55.7 mmol) at room temperature over 15 minutes. The reaction mixture was stirred at 75° C. for 12 hours. It was cooled to room temperature, extracted with ethyl acetate, washed with brine, dried over Na₂SO₄ and purified by silica gel column chromatography (petroleum ether:ethyl acetate=20:1) to give the title compound (5.45 g, 95% purity from ¹H NMR, 26% yield) as yellow solids. LC-MS (ESI): RT=1.74 min, mass calcd. for C₁₇H₁₅ClFN₃O₂S 379.1, m/z found 380.1 [M+H]⁺. ¹H NMR (400 MHz, CDCl₃) δ 7.84-7.80 (m, 1.7H), 7.50 (d, J=3.6 Hz, 0.3H), 7.47 (s, 0.3H), 7.44 (d, J=3.2 Hz, 0.7H), 7.23-7.14 (m, 2H), 7.09-7.01 (m, 1H), 6.27 (s, 0.7H), 6.14 (d, J=2.4 Hz, 0.3H), 4.13-3.98 (m, 2H), 2.57 (s, 0.7H), 2.52 (s, 2.3H), 1.13-1.10 (m, 3H).

The racemic mixture ethyl 4-(2-chloro-3-fluorophenyl)-6-methyl-2-(thiazol-2-yl)-1,4-dihydropyrimidine-5-carboxylate 111 (5.45 g, 95% purity, 13.7 mmol) was separated by chiral separation (separation condition: column: Chiralpak IC 5 μm 20*250 mm; Mobile Phase:Hex:EtOH:DEA=95:5:0.3 at 28 mL/min, Temp: 30° C., Wavelength: 254 nm) to give the title compounds H1-A (2.5 g, 90% purity from ¹HNMR, 46% yield, 100% ee) and H1-B (2.48 g, 90% purity from ¹HNMR, 46% yield, 92.1% ee) as yellow solids. H1-A: LC-MS (ESI): R_{T}=3.886 min, mass calcd. for C₁₇H₁₅ClFN₃O₂S 379.06, m/z found 380.1 [M+H]⁺. Chiral analysis (Column: Chiralpak IA 5 μm 4.6*250 mm; Mobile Phase: Hex:EtOH:DEA=90:10:0.2 at 1.0 mL/min; Temp: 30° C.; Wavelength: 254 nm, R_{T}=7.438 min). ¹H NMR (400 MHz, CDCl₃) δ 7.84-7.80 (m, 1.7H), 7.51-7.44 (m, 1.3H), 7.22-7.14 (m, 2H), 7.09-7.01 (m, 1H), 6.27 (s, 0.7H), 6.14 (s, 0.3H), 4.05-4.00 (m, 2H), 2.57 (s, 0.7H), 2.52 (s, 2.3H), 1.13-1.10 (m, 3H).

H1-B: LC-MS (ESI): R_{T}=3.887 min, mass calcd. for C₁₇H₁₅ClFN₃O₂S 379.06, m/z found 380.1 [M+H]⁺. Chiral analysis (Column: Chiralpak IA 5 μm 4.6*250 mm; Mobile Phase:Hex:EtOH:DEA=90:10:0.2 at 1.0 mL/min; Temp: 30° C.; Wavelength: 254 nm, R_{T}=6.903 min). ¹H NMR (400 MHz, CDCl₃) δ 7.84-7.80 (m, 1.7H), 7.51-7.43 (m, 1.3H), 7.22-7.14 (m, 2H), 7.09-7.01 (m, 1H), 6.27 (s, 0.7H), 6.14 (s, 0.3H), 4.10-3.98 (m, 2H), 2.57 (s, 0.7H), 2.51 (s, 2.3H), 1.13-1.10 (m, 3H).

### Intermediate H1-1A: (R*)-Ethyl 6-(bromomethyl)-4-(2-chloro-3-fluorophenyl)-2-(thiazol-2-yl)-1,4-dihydropyrimidine-5-carboxylate

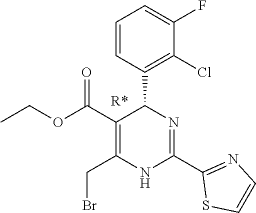 [SMILES: CCOC(=O)C1=C(CBr)NC(c2nccs2)=N[C@H]1c1cccc(F)c1Cl]

To a solution of (R*)-ethyl 4-(2-chloro-3-fluorophenyl)-6-methyl-2-(thiazol-2-yl)-1,4-dihydropyrimidine-5-carboxylate H1-A (300 mg, 90% purity, 0.711 mmol) in carbon tetrachloride (5 mL) was added N-bromosuccinimide (120 mg, 0.674 mmol). After stirred at 60° C. for 1 hour, the reaction mixture was concentrated to give a residue, which was purified by gel column chromatography (petroleum ether:ethyl acetate=20:1 to 10:1) to give the title compound (240 mg, 90% purity from HNMR, 66% yield) as yellow solids. LC-MS (ESI): R_{T}=1.852 min, mass calcd. for C₁₇H₁₄BrClFN₃O₂S 456.9, m/z found 457.9 [M+H]⁺. ¹H NMR (400 MHz, CDCl₃) δ 8.26 (s, 0.3H), 7.84 (d, J=2.8 Hz, 1H), 7.53-7.46 (m, 1.7H), 7.24-7.14 (m, 2H), 7.09-7.01 (m, 1H), 6.26 (s, 0.3H), 6.17 (s, 0.7H), 4.92 (d, J=8.0 Hz, 1H), 4.76 (d, J=11.2 Hz, 0.3H), 4.60 (d, J=8.0 Hz, 0.7H), 4.12 (q, J=7.2 Hz, 2H), 1.14 (t, J=11.2 Hz, 3H).

### Intermediate H2: Ethyl 4-(3-fluoro-2-methylphenyl)-6-methyl-2-(thiazol-2-yl)-1,4-dihydropyrimidine-5-carboxylate

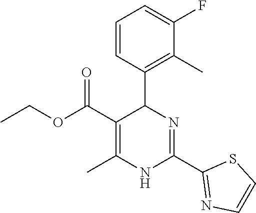 [SMILES: CCOC(=O)C1=C(C)NC(c2nccs2)=NC1c1cccc(F)c1C]

To a mixture of 3-fluoro-2-methylbenzaldehyde (4.00 g, 28.9 mmol), ethyl acetoacetate (3.77 g, 28.9 mmol) and thiazole-2-carboximidamide hydrochloride (4.74 g, 28.9 mmol) in methanol (50 mL) was added sodium acetate (2.37 g, 28.9 mmol) at room temperature. After stirred at 80° C. overnight, the reaction mixture was cooled down to room temperature and concentrated to give a residue, which was purified by silica gel column chromatography (petroleum ether:ethyl acetate=10:1 to 1:1) to give the title compound (6.00 g, 58% yield) as yellow solids. ¹H NMR (400 MHz, DMSO-d₆) δ 9.86 (s, 0.8H), 9.52 (d, J=2.8 Hz, 0.2H), 8.00-7.98 (m, 0.4H), 7.96 (d, J=3.2 Hz, 0.8H), 7.88 (d, J=2.8 Hz, 0.8H), 7.20-7.15 (m, 1.2H), 7.06-6.99 (m, 1.8H), 5.83 (s, 0.8H), 5.73 (d, J=3.2 Hz, 0.2H), 3.99-3.93 (m, 2H), 2.48 (s, 2.4H), 2.45 (s, 1.2H), 2.44 (s, 1.2H), 2.41 (s, 0.3H), 2.40 (s, 0.3H), 2.37 (s. 0.6H), 1.08-1.02 (m, 3H).

Intermediate H2 was separated by chiral Prep. HPLC (separation condition: Column: Chiralpak OJ-H 5 μm 20*250 mm; Mobile Phase:Hex:EtOH:DEA=90:10:0.3 at 15 mL/min; Temp: 30° C.; Wavelength: 214 nm) to afford intermediate H2-A and intermediate H2-B as yellow solids.

Intermediate H2-A: Chiral analysis (Column: Chiralpak OJ-H 5 μm 4.6*250 mm; Mobile Phase:Hex:EtOH:DEA=85:15:0.2 at 1.0 mL/min; Temp: 30° C.; Wavelength: 230 nm, R_{T}=7.251 min). H2-A was certificated to absolute S stereochemistry by the following chemical resolution which is consistent with reported data (J. Med. Chem., 2017, 60 (8), pp 3352-3371). Optical rotation: [a]_{D}²⁰=24° (c 0.10, MeOH).

Intermediate H2-B: Chiral analysis (Column: Chiralpak OJ-H 5 μm 4.6*250 mm; Mobile Phase:Hex:EtOH:DEA=85:15:0.2 at 1.0 mL/min; Temp: 30° C.; Wavelength: 230 nm, R_{T}=9.072 min). Optical rotation: [a]_{D}²⁰=35° (c 0.10, MeOH).

### Intermediate H2-1A: (S)-Ethyl 6-(bromomethyl)-4-(3-fluoro-2-methylphenyl)-2-(thiazol-2-yl)-1,4-dihydropyrimidine-5-carboxylate

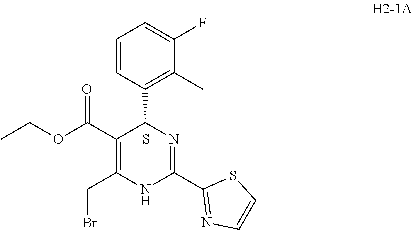

To the solution of (S)-ethyl 4-(3-fluoro-2-methylphenyl)-6-methyl-2-(thiazol-2-yl)-1,4-dihydropyrimidine-5-carboxylate H2-A (10 g, 99% purity, 27.6 mmol) in carbon tetrachloride (300 mL) was added N-bromo succinimide (4.9 g, 27.5 mmol) at room temperature under nitrogen atmosphere. After refluxing at room temperature overnight under atmosphere, the mixture was concentrated under reduced pressure to give a residue, which was diluted in ethyl acetate (100 mL) and washed with water (50 mL) twice, then the combined aqueous layers were extracted with ethyl acetate (50 mL) twice. The combined organic layers were washed with water (20 mL) twice and brine (20 mL), dried over Na₂SO₄₍ₛ₎ and filtered. The filtrate was concentrated under reduced pressure to afford the residue, which was purified by silica gel column chromatography (petroleum ether:ethyl acetate=10:1 to 5:1) to give the title compound (6.5 g, 95% purity form NMR, 51% yield) as yellow solids. LC-MS (ESI): R_{T}=1.84 min, mass calcd. for C₁₈H₁₇BrFN₃O₂S 437.0, m/z found 440.0 [M+H]⁺. ¹H NMR (400 MHz, CDCl₃) δ 8.22 (s, 0.5H), 7.82 (d, J=3.2 Hz, 1H), 7.53 (s, 0.4H), 7.44 (s, 0.6H), 7.25-7.08 (m, 2.5H), 6.96-6.92 (s, 1H), 5.99 (s, 0.6H), 5.93 (s, 0.4H), 4.92-4.77 (m, 1.6H), 4.67-4.65 (m, 0.4H), 4.13-4.07 (m, 2H), 2.53 (s, 1.7H), 2.41 (s, 1.3H), 1.14 (t, J=7.2 Hz, 3H). Optical rotation: [a]_{D}²⁰+0.093° (c 0.10, MeOH).

### Intermediate H3: Methyl 4-(2-bromo-4-fluorophenyl)-6-methyl-2-(thiazol-2-yl)-1,4-dihydropyrimidine-5-carboxylate

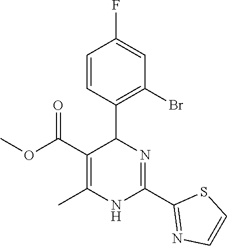 [SMILES: COC(=O)C1=C(C)NC(c2nccs2)=NC1c1ccc(F)cc1Br]

To a solution of methyl 3-oxobutanoate (1.0 g, 8.61 mmol), 2-bromo-4-fluorobenzaldehyde (1.75 g, 8.62 mmol) and thiazole-2-carboximidamide hydrochloride (1.41 g, 8.62 mmol) in methanol (10 mL) was added sodium acetate (706 mg, 8.61 mmol) at room temperature under nitrogen atmosphere. After stirring at 80° C. overnight, the mixture was allowed to cool down to room temperature and filtered. The filtrate was concentrated under reduced pressure to give a residue, which was purified by silica gel column chromatography (petroleum ether:ethyl acetate:tetrahydrofuran=10:1:1), then further purified by C18 column (acetonitrile:water=20% to 95%) to give the title compound (1.80 g, 90% purity from ¹H NMR, 46% yield) as yellow solids. ¹H NMR (400 MHz, CDCl₃) δ 7.89-7.75 (m, 1.7H), 7.62-7.55 (m, 0.3H), 7.49-7.40 (m, 1H), 7.33-7.29 (m, 2H), 7.00-6.94 (m, 1H), 6.15 (s, 0.7H), 6.03 (s, 0.3H), 3.61 (s, 3H), 2.52 (s, 3H).

A racemic mixture of methyl 4-(2-bromo-4-fluorophenyl)-6-methyl-2-(thiazol-2-yl)-1,4-dihydropyrimidine-5-carboxylate H3 (1.80 g, 90% purity, 3.95 mmol) was separated by chiral Prep. HPLC (Column: Chiralpak IG 5 μm 20 mm*250 mm; Mobile Phase: CO₂: MeOH=75:25 at 50 g/min; Col. Temp: 40° C.; Wavelength: 230 nm, Back pressure: 100 bar) to afford the title compounds H3-A (850 mg, 90% purity from ¹H NMR, 47% yield, 99.6% ee) and H3-B (850 mg, 90% purity from ¹H NMR, 47% yield, 99.4% ee) as yellow solids.

H3-A: LC-MS (ESI): R_{T}=1.717 min, mass calcd. for C₁₆H₁₃BrFN₃O₂S 409.0, m/z found 410.0 [M+H]⁺. Chiral analysis (Column: Chiralpak IG 5 μm 4.6*250 mm; Mobile Phase: CO₂: MeOH=75:25 at 3 g/min; Temp: 40° C.; Wavelength: 230 nm; Back pressure: 100 bar, R_{T}=3.92 min). ¹H NMR (400 MHz, CDCl₃) δ 7.87-7.84 (m, 1H), 7.80 (d, J=3.2 Hz, 0.7H), 7.57 (br s, 0.3H), 7.51 (d, J=3.2 Hz, 0.3H), 7.44 (d, J=3.2 Hz, 0.7H), 7.34-7.29 (m, 2H), 7.01-6.93 (m, 1H), 6.16 (s, 0.7H), 6.02 (d, J=2.4 Hz, 0.3H), 3.62 (s, 1H), 3.60 (s, 2H), 2.57 (s, 1H), 2.51 (s, 2H).

H3-B: LC-MS (ESI): R_{T}=1.713 min, mass calcd. for C₁₆H₁₃BrFN₃O₂S 409.0, m/z found 410.0 [M+H]⁺. Chiral analysis (Column: Chiralpak IG 5 μm 4.6*250 mm; Mobile Phase: CO₂:MeOH=75:25 at 3 g/min; Temp: 40° C.; Wavelength: 230 nm; Back pressure: 100 bar, R_{T}=4.92 min). ¹H NMR (400 MHz, CDCl₃) δ 7.88-7.83 (m, 1H), 7.80 (d, J=3.2 Hz, 0.7H), 7.58 (br s, 0.3H), 7.50 (d, J=3.2 Hz, 0.3H), 7.44 (d, J=3.2 Hz, 0.7H), 7.34-7.29 (m, 2H), 7.01-6.93 (m, 1H), 6.16 (s, 0.7H), 6.02 (d, J=2.0 Hz, 0.3H), 3.62 (s, 1H), 3.60 (s, 2H), 2.57 (s, 1H), 2.51 (s, 2H).

### Intermediate H3-1A: (R 1-Methyl 4-(2-bromo-4-fluorophenyl)-6-(bromomethyl)-2-(thiazol-2-yl)-1,4-dihydropyrimidine-5-carboxylate

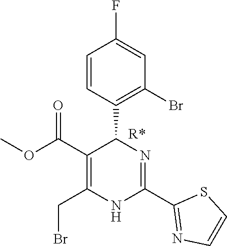 [SMILES: COC(=O)C1=C(CBr)NC(c2nccs2)=N[C@H]1c1ccc(F)cc1Br]

To the solution of (R*)-methyl 4-(2-bromo-4-fluorophenyl)-6-methyl-2-(thiazol-2-yl)-1,4-dihydropyrimidine-5-carboxylate H3-A (300 mg, 90% purity, 0.658 mmol) in perchloromethane (10 mL) was added 1-bromopyrrolidine-2,5-dione (129 mg, 0.725 mmol) at room temperature under nitrogen atmosphere. After stirred at room temperature overnight, the mixture was cooled to room temperature and diluted with water (20 mL) and extracted with dichloromethane (20 mL) for three times. The combined organic layers were washed with brine (20 mL), dried over Na₂SO₄₍ₛ₎ and filtered. The filtrate was concentrated and purified by silica gel column chromatography (petroleum ether:ethyl acetate=20:1 to 10:1) to give the title compound (210 mg, 90% purity from ¹H NMR, 59% yield) as yellow solids. ¹H NMR (400 MHz, CDCl₃) δ 7.85 (d, J=3.2 Hz, 1H), 7.52 (d, J=2.8 Hz, 1H), 7.40-7.36 (m, 1H), 7.34-7.32 (m, 1H), 7.04-6.99 (m, 1H), 6.09 (s, 1H), 4.95 (d, J=9.2 Hz, 1H), 4.63 (d, J=8.4 Hz, 1H), 3.67 (s, 3H).

### Preparation of Intermediate S1

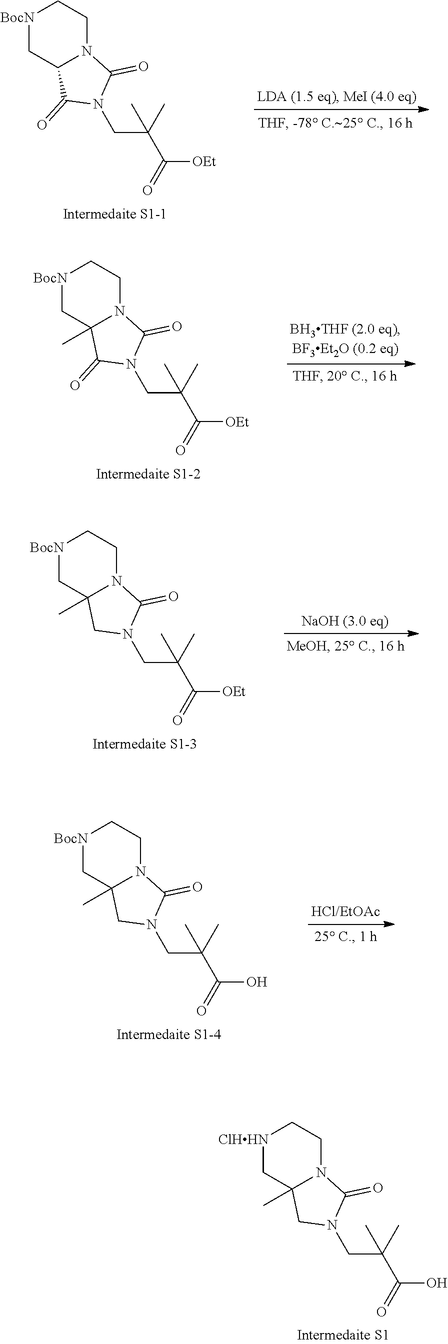

### Intermediate S1-2: tert-Butyl 2-(3-ethoxy-2,2-dimethyl-3-oxopropyl)-8a-methyl-1,3-dioxohexahydroimidazo[1,5-a]pyrazine-7(1H)-carboxylate

To a solution of (S)-tert-butyl 2-(3-ethoxy-2,2-dimethyl-3-oxopropyl)-1,3-dioxohexa hydroimidazo[1,5-a]pyrazine-7(1H)-carboxylate S1-1 (3.00 g, 7.04 mmol, Cas #2126690-40-0) in tetrahydrofuran (30 mL) was added 1 M lithium bis(trimethylsilyl)amide in THF (10.8 mL, 10.8 mmol) at −78° C. under nitrogen atmosphere and stirred at this temperature for 1 hour. Iodomethane (4.00 g, 28.2 mmol) was added and the reaction mixture was stirred at 25° C. for another 16 hours. The reaction mixture was quenched with saturated ammonium chloride (50 mL) dropwise. The organic phase was separated and the aqueous layer was extracted with ethyl acetate (20 mL) for three times. The combined organic phases were dried over Na₂SO₄₍ₛ₎ and filtered. The filtrate was concentrated to give a residue, which was purified by column chromatography (petroleum ether:ethyl acetate=10:1 to 4:1) to give the title compound (2.61 g, 84% yield) as yellow oil. LC-MS (ESI): R_{T}=1.653 min, mass calcd. for C₁₉H₃₁N₃O₆ 397.2, m/z found 398.2 [M+H]⁺. ¹H NMR (400 MHz, CDCl₃) δ 4.29-4.07 (m, 4H), 4.01 (dd, J=14.0, 3.6 Hz, 1H), 3.69 (d, J=14.0 Hz, 1H), 3.65 (d, J=14.0 Hz, 1H), 3.11-2.99 (m, 1H), 2.85-2.62 (m, 2H), 1.48 (s, 9H), 1.45 (s, 3H), 1.28 (t, J=7.2 Hz, 3H), 1.21 (s, 6H).

### Intermediate S1-3: tert-Butyl 2-(3-ethoxy-2,2-dimethyl-3-oxopropyl)-8a-methyl-3-oxohexahydroimidazo[1,5-a]pyrazine-7(1H)-carboxylate

To a solution of tert-butyl 2-(3-ethoxy-2,2-dimethyl-3-oxopropyl)-8a-methyl-1,3-diox ohexahydroimidazo[1,5-a]pyrazine-7(1H)-carboxylate S1-2 (2.61 g, 5.91 mmol) in tetrahydrofuran (6 mL) was added boron trifluoride etherate (168 mg, 1.18 mmol) at 0° ˜5° C. and followed with adding 1 M Borane-tetrahydrofuran complex (12 mL, 12 mmol) slowly at 5° C.˜10° C. over 10 minutes. The reaction mixture was stirred at 20° C. for another 16 hours under nitrogen atmosphere. The reaction mixture was quenched with ethyl acetate (50 mL) and 3% wt aqueous sodium carbonate (30 mL) and stirred for 30 minutes. The mixture was separated and the aqueous layer was extracted with ethyl acetate (30 mL) for three times. The combined organic phases were dried over Na₂SO₄₍ₛ₎ and filtered. The filtrate was concentrated to give a residue, which was purified by column chromatography (petroleum ether:ethyl acetate=10:1 to 7:3) to give the title compound (660 mg, 26% yield) as colorless oil. LC-MS (ESI): R_{T}=1.630 min, mass calcd. for C₁₉H₃₃N₃O₅ 383.2, m/z found 384.2 [M+H]⁺. ¹H NMR (400 MHz, CDCl₃) δ 4.15 (q, J=7.2 Hz, 2H), 4.07-3.88 (m, 2H), 3.72 (dd, J=13.6, 3.2 Hz, 1H), 3.45 (d, J=14.4 Hz, 1H), 3.27 (d, J=14.0 Hz, 1H), 3.10-2.98 (m, 3H), 2.90-2.62 (m, 2H), 1.46 (s, 9H), 1.29-1.26 (m, 6H), 1.21 (s, 3H), 1.20 (s, 3H). Racemic S1-3 (500 mg, 1.17 mmol) was separated by chiral Prep. HPLC (Column: Chiralpak IG 5 μm 20*250 mm; Mobile Phase:MeOH:EtOH=60:40 at 18 mL/min; Temp: 30° C.; Wavelength: 214 nm) to give the title compound S1-3A (210 mg, 100% ee) as white solids and the title compound S1-3B (220 mg, 100% ee) as white solids.

Intermediate S1-3A: LC-MS (ESI): R_{T}=1.634 min, mass calcd. for C₁₉H₃₃N₃O₅ 383.2, m/z found 384.2[M+H]⁺. Chiral analysis (Column: Chiralpak IG 5 μm 4.6*250 mm; Mobile Phase:MeOH:EtOH=50:50 at 1.0 mL/min; Temp: 30° C.; Wavelength: 230 nm, R_{T}=5.721 min). ¹H NMR (400 MHz, CDCl₃) δ 4.14 (q, J=7.2 Hz, 2H), 4.09-3.81 (m, 2H), 3.71 (d, J=13.2 Hz, 1H), 3.45 (d, J=14.0 Hz, 1H), 3.26 (d, J=14.0 Hz, 1H), 3.10-2.97 (m, 3H), 2.90-2.61 (m, 2H), 1.46 (s, 9H), 1.29-1.26 (m, 6H), 1.21 (s, 3H), 1.20 (s, 3H).

Intermediate S1-3B: Chiral analysis (Column: Chiralpak IG 5 μm 4.6*250 mm; Mobile Phase:MeOH:EtOH=50:50 at 1.0 mL/min; Temp: 30° C.; Wavelength: 230 nm, R_{T}=12.205 min). LC-MS (ESI): R_{T}=1.660 min, mass calcd. for C₁₉H₃₃N₃O₅ 383.2, m/z found 384.3[M+H]⁺. ¹H NMR (400 MHz, CDCl₃) δ 4.15 (q, J=7.2 Hz, 2H), 4.08-3.78 (m, 2H), 3.71 (d, J=13.6 Hz, 1H), 3.45 (d, J=14.4 Hz, 1H), 3.26 (d, J=14.0 Hz, 1H), 3.10-2.98 (m, 3H), 2.90-2.60 (m, 2H), 1.46 (s, 9H), 1.29-1.26 (m, 6H), 1.21 (s, 3H), 1.20 (s, 3H).

### Intermediate S1-4: 3-(7-(tert-Butoxycarbonyl)-8a-methyl-3-oxohexahydroimidazo[1,5-a]pyrazin-2(3H)-yl)-2,2-dimethylpropanoic Acid

To a solution of tert-butyl 2-(3-ethoxy-2,2-dimethyl-3-oxopropyl)-8a-methyl-3-oxohexahydroimidazo[1,5-a]pyrazine-7(1H)-carboxylate S1-3 (240 mg, 0.563 mmol) in methanol (4 mL) was added sodium hydroxide (68 mg, 1.7 mmol) in water (1 mL). After stirred at 25° C. for 16 hours, the reaction mixture was diluted with water (10 mL) and extracted with ethyl acetate (5 mL). The aqueous layer was acidified with 3 M hydrochloride aqueous solution to pH 3-4 and extracted with ethyl acetate (10 mL) for three times. The combined organic layer was washed with brine (5 mL), dried over Na₂SO₄₍ₛ₎, and filtered. The filtrate was concentrated to give the title compound (180 mg, 81% yield) as white solids. LC-MS (ESI): R_{T}=1.161 min, mass calcd. for C₁₇H₂₉N₃O₅ 355.2, m/z found 356.2 [M+H]⁺. ¹H NMR (400 MHz, CDCl₃) δ 4.18-3.81 (m, 2H), 3.72 (d, J=12.8 Hz, 1H), 3.49 (d, J=14.0 Hz, 1H), 3.28 (d, J=14.0 Hz, 1H), 3.17 (s, 2H), 3.02 (t, J=11.6 Hz, 1H), 2.88-2.65 (m, 2H), 1.46 (s, 9H), 1.31 (s, 3H), 1.24 (s, 6H).

### Intermediate S1-4A: 3-(7-(tert-butoxycarbonyl)-8a-methyl-3-oxohexahydroimidazo[1,5-a]pyrazin-2(3H)-yl)-2,2-dimethylpropanoic Acid

S1-4A was prepared from S1-3A using same condition as for S1-4. LC-MS (ESI): R_{T}=1.175 min, mass calcd. for C₁₇H₂₉N₃O₅ 355.2, m/z found 356.2 [M+H]⁺. ¹H NMR (400 MHz, CDCl₃) δ 4.15-3.79 (m, 2H), 3.72 (dd, J=13.6, 3.2 Hz, 1H), 3.49 (d, J=14.4 Hz, 1H), 3.27 (d, J=14.4 Hz, 1H), 3.17 (s, 2H), 3.02 (t, J=11.2 Hz, 1H), 2.90-2.62 (m, 2H), 1.46 (s, 9H), 1.31 (s, 3H), 1.25 (s, 3H), 1.24 (s, 3H).

### Intermediate S1: 2,2-Dimethyl-3-(8a-methyl-3-oxohexahydroimidazo[1,5-a]pyrazin-2(3H)-yl)propanoic Acid Hydrochloride

A solution of 3-(7-(tert-butoxycarbonyl)-8a-methyl-3-oxohexahydroimidazo[1,5-a]pyrazin-2 (3H)-yl)-2,2-dimethylpropanoic acid S1-4 (180 mg, 0.456 mmol) in 2 M hydrochloride in ethyl acetate (4 mL) was stirred at 25° C. for 1 hour. The reaction mixture was concentrated to give the title compound (120 mg, 81% yield) as white solids. LC-MS (ESI): R_{T}=0.238 min, mass calcd. for Chemical Formula: C₁₂H₂₂ClN₃O₃ 291.1, m/z found 256.2 [M−HCl+H]⁺. ¹H NMR (400 MHz, DMSO-d₆) δ 9.93-9.90 (m, 1H), 9.60-9.44 (m, 1H), 3.67 (dd, J=14.8, 2.8 Hz, 1H), 3.30 (d, J=14.0 Hz, 1H), 3.23-3.15 (m, 4H), 3.11-3.06 (m, 2H), 2.78 (t, J=11.6 Hz, 1H), 2.67-2.55 (m, 1H), 1.49 (s, 3H), 1.09 (s, 3H), 1.08 (s, 3H).

### Intermediate S1-A: 2,2-Dimethyl-3-(8a-methyl-3-oxohexahydroimidazo[1,5-a]pyrazin-2(3H)-yl)propanoic Acid Hydrochloride

S1-A was prepared from S1-4A using same condition as for S1. ¹H NMR (400 MHz, DMSO-d₆) δ 10.1 (br s, 1H), 3.66 (dd, J=14.4, 3.2 Hz, 1H), 3.30 (d, J=14.4 Hz, 1H), 3.23-3.14 (m, 4H), 3.10-3.06 (m, 2H), 2.77 (d, J=12.4 Hz, 1H), 2.64-2.56 (m, 1H), 1.49 (s, 3H), 1.09 (s, 3H), 1.07 (s, 3H).

### Preparation of intermediate S2-A (S*)-3-(8a-Ethyl-3-oxohexahydroimidazo[1,5-a]pyrazin-2(3H)-yl)-2,2-dimethylpropanoic Acid Hydrochloride

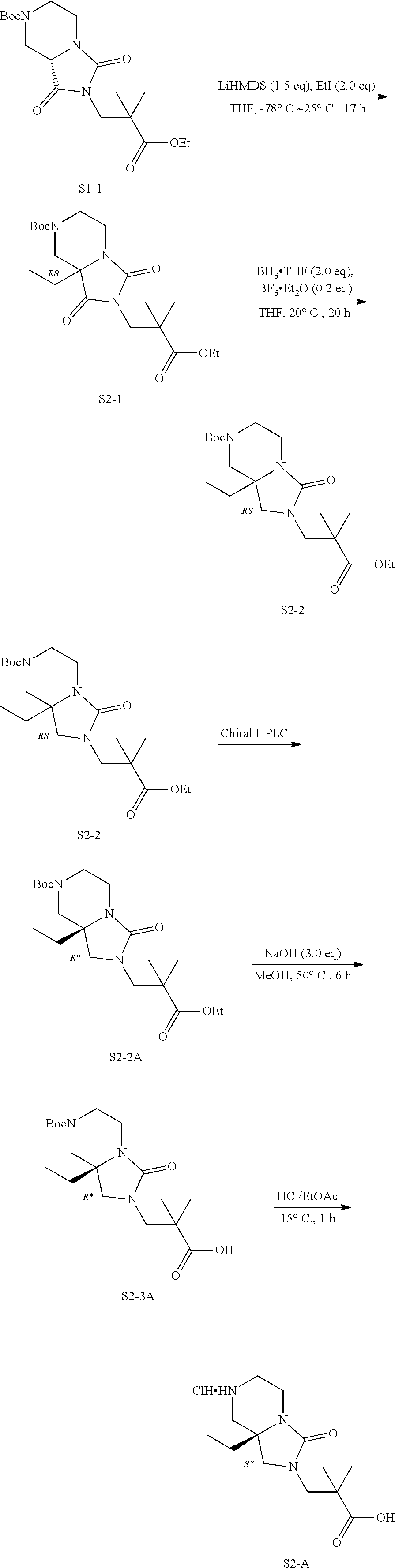

### Intermediate S2-1: tert-Butyl 2-(3-ethoxy-2,2-dimethyl-3-oxopropyl)-8a-ethyl-1,3-dioxohexahydroimidazo[1,5-a]pyrazine-7(1H)-carboxylate

To a solution of (S)-tert-butyl 2-(3-ethoxy-2,2-dimethyl-3-oxopropyl)-1,3-dioxohexahydroimidazo[1,5-a]pyrazine-7(1H)-carboxylate S1-1 (2 g, 90% purity, 4.69 mmol) in tetrahydrofuran (20 mL) was added 1 M lithium bis(trimethylsilyl)amide in tetrahydrofuran (7.2 mL, 7.2 mmol) at −78° C. under nitrogen atmosphere and stirred at this temperature for 1 hour. iodoethane (1.5 g, 9.62 mmol) was added and the reaction mixture was stirred at 25° C. for another 16 hours. The reaction mixture was quenched with saturated ammonium chloride (50 mL) dropwise. The organic phase was separated and the aqueous layer was extracted with ethyl acetate (30 mL) for three times. The combined organic phases were dried over Na₂SO₄₍ₛ₎ and filtered. The filtrate was concentrated to give a residue, which was purified by gel column chromatography (petroleum ether:ethyl acetate=10:1 to 4:1) and further purified by C-18 (acetonitrile:water=30% to 60%) to give the title compound (920 mg, 90% purity from HNMR, 43% yield) as yellow oil. ¹H NMR (400 MHz, CDCl₃) δ 4.30-4.16 (m, 2H), 4.13 (q, J=7.2 Hz, 2H), 4.04 (dd, J=13.6, 3.6 Hz, 1H), 3.71 (d, J=14.0 Hz, 1H), 3.65 (d, J=13.6 Hz, 1H), 2.98-2.92 (m, 1H), 2.85-2.62 (m, 2H), 1.91 (q, J=7.2 Hz, 2H), 1.47 (s, 9H), 1.28 (t, J=7.2 Hz, 3H), 1.21 (s, 3H), 1.20 (s, 3H) 0.83 (t, J=7.2 Hz, 3H).

### Intermediate S2-2: tert-Butyl 2-(3-ethoxy-2,2-dimethyl-3-oxopropyl)-8a-ethyl-3-oxohexahydroimidazo[1,5-a]pyrazine-7(1H)-carboxylate

To a solution of tert-butyl 2-(3-ethoxy-2,2-dimethyl-3-oxopropyl)-8a-ethyl-1,3-dioxohexahydroimidazo[1,5-a]pyrazine-7(1H)-carboxylate S2-1 (1.65 g, 90% purity, 3.61 mmol) in tetrahydrofuran (4 mL) was added boron trifluoride etherate (103 mg, 0.726 mmol) at 0° C.˜5° C. and followed with adding 1 M borane-tetrahydrofuran complex (7.4 mL, 7.4 mmol) slowly at 5° C.˜10° C. over 10 minutes. The reaction mixture was stirred at 15° C. for another 20 hours under nitrogen atmosphere. The reaction mixture was quenched with ethyl acetate (50 mL) and 3% wt aqueous sodium carbonate (30 mL) below 10° C. and stirred for 30 minutes. The phases were separated and the aqueous layer was extracted with ethyl acetate (20 mL) for three times. The combined organic phases were dried over Na₂SO₄₍ₛ₎ and filtered. The filtrate was concentrated to give a residue, which was purified by gel column chromatography (petroleum ether:ethyl acetate=8:1 to 4:1) and further purified by C-18 (acetonitrile:water=30% to 55%) to give compound (210 mg, 90% purity from HNMR, 13.2% yield) as colorless oil. LC-MS (ESI): R_{T}=1.686 min, mass calcd. for C₂₀H₃₅N₃O₅ 397.5, m/z found 398.3 [M+H]⁺. ¹H NMR (400 MHz, CDCl₃) δ 4.15 (q, J=7.2 Hz, 2H), 4.06-3.83 (m, 2H), 3.74 (dd, J=13.2, 2.8 Hz, 1H), 3.45 (d, J=14.0 Hz, 1H), 3.28 (d, J=14.0 Hz, 1H), 3.13 (d, J=9.2 Hz, 1H), 2.98-2.85 (m, 2H), 2.81-2.62 (m, 2H), 1.75-1.65 (m, 2H), 1.46 (s, 9H), 1.28 (t, J=7.2 Hz, 3H), 1.21 (s, 3H), 1.20 (s, 3H), 0.86 (t, J=7.2 Hz, 3H).

### Intermediates S2-2A and S2-2B

### (R*)-tert-Butyl 2-(3-ethoxy-2,2-dimethyl-3-oxopropyl)-8a-ethyl-3-oxohexahydroimidazo pyrazine-7(1H)-carboxylate and (S*)-tert-butyl 2-(3-ethoxy-2,2-dimethyl-3-oxopropyl)-8a-ethyl-3-oxohexahydroimidazo pyrazine-7(1H)-carboxylate

A racemic of tert-butyl 2-(3-ethoxy-2,2-dimethyl-3-oxopropyl)-8a-ethyl-3-oxohexahydroimidazo[1,5-a]pyrazine-7(1H)-carboxylate S2-2 (320 mg, 90% purity, 0.725 mmol) was separated by chiral prep. HPLC (separation condition: Column: Chiralpak IC 5 μm 20*250 mm; Mobile Phase:Hex:IPA=60:40 at 15 mL/min; Temp: 30° C.; Wavelength: 214 nm) to give the title compound S2-2A (150 mg, 90% purity from HNMR, 46.9% yield, 100% ee) as white solids and the title compound S2-2B (130 mg, 90% purity from HNMR, 40.6% yield, 99.8% ee) as white solids.

### Intermediate S2-2A

LC-MS (ESI): R_{T}=1.721 min, mass calcd. for C₂₀H₃₅N₃O₅ 397.3, m/z found 398.3[M+H]⁺. Chiral analysis (Column: Chiralpak IC 5 μm 4.6*250 mm; Mobile Phase:Hex:IPA=60:40 at 1.0 mL/min; Temp: 30° C.; Wavelength: 214 nm, R_{T}=9.611 min). ¹H NMR (400 MHz, CDCl₃) δ 4.15 (q, J=7.2 Hz, 2H), 4.09-3.83 (m, 2H), 3.73 (dd, J=13.2, 2.4 Hz, 1H), 3.45 (d, J=14.0 Hz, 1H), 3.28 (d, J=14.0 Hz, 1H), 3.13 (d, J=8.8 Hz, 1H), 2.92-2.90 (m, 2H), 2.84-2.62 (m, 2H), 1.63-1.52 (m, 2H), 1.46 (s, 9H), 1.28 (t, J=7.2 Hz, 3H), 1.21 (s, 3H), 1.20 (s, 3H), 0.86 (t, J=7.2 Hz, 3H).

### Intermediate S2-2B

LC-MS (ESI): R_{T}=1.726 min, mass calcd. for C₂₀H₃₅N₃O₅ 397.3, m/z found 398.2[M+H]⁺. Chiral analysis (Column: Chiralpak IC 5 μm 4.6*250 mm; Mobile Phase:Hex:IPA=60:40 at 1.0 mL/min; Temp: 30° C.; Wavelength: 214 nm, R_{T}=11.96 min). ¹H NMR (400 MHz, CDCl₃) δ 4.15 (q, J=7.2 Hz, 2H), 4.04-3.87 (m, 2H), 3.73 (dd, J=13.2, 2.8 Hz, 1H), 3.45 (d, J=14.0 Hz, 1H), 3.28 (d, J=14.0 Hz, 1H), 3.13 (d, J=9.2 Hz, 1H), 2.92-2.90 (m, 2H), 2.85-2.64 (m, 2H), 1.64-1.53 (m, 2H), 1.46 (s, 9H), 1.28 (t, J=7.2 Hz, 3H), 1.21 (s, 3H), 1.20 (s, 3H), 0.86 (t, J=7.2 Hz, 3H).

### Intermediate S2-3A: (R*)-3-(7-(tert-Butoxycarbonyl)-8a-ethyl-3-oxohexahydroimidazo[1,5-a]pyrazin-2(3H)-yl)-2,2-dimethylpropanoic Acid

To a solution of (R*)-tert-butyl 2-(3-ethoxy-2,2-dimethyl-3-oxopropyl)-8a-ethyl-3-oxohexahydroimidazo[1,5-a]pyrazine-7(1H)-carboxylate S2-2A (150 mg, 90% purity, 0.34 mmol) in methanol (4 mL) was added sodium hydroxide (41 mg, 1.03 mmol) in water (1 mL). After stirred at 50° C. for 6 hours, the reaction mixture was diluted with water (20 mL) and concentrated methanol under vacuum, then extracted with ethyl acetate (5 mL). The aqueous layer was acidified with 3 M hydrochloride aqueous solution to pH 3˜4 and extracted with ethyl acetate (10 mL) for three times. The combined organic layer was washed with brine (5 mL), dried over Na₂SO₄₍ₛ₎ and filtered. The filtrate was concentrated to give the title compound (130 mg, 90% purity from HNMR, 93.2% yield) as white solids. LC-MS (ESI): R_{T}=1.239 min, mass calcd. for C₁₈H₃₁N₃O₅ 369.2, m/z found 370.2 [M+H]⁺. ¹H NMR (400 MHz, CDCl₃) δ 4.08-3.85 (m, 2H), 3.76-3.71 (m, 1H), 3.49 (d, J=14.4 Hz, 1H), 3.28 (d, J=14.0 Hz, 1H), 3.22 (d, J=9.2 Hz, 1H), 3.02 (d, J=9.6 Hz, 1H), 2.97-2.64 (m, 3H), 1.77-1.55 (m, 2H), 1.46 (s, 9H), 1.25 (s, 3H), 1.24 (s, 3H), 0.86 (t, J=7.2 Hz, 3H).

### Intermediate S2-A: (S*)-3-(8a-Ethyl-3-oxohexahydroimidazo[1,5-a]pyrazin-2(3H)-yl)-2,2-dimethylpropanoic Acid Hydrochloride

A solution of (R*)-3-(7-(tert-butoxycarbonyl)-8a-ethyl-3-oxohexahydroimidazo[1,5-a]pyrazin-2(3H)-yl)-2,2-dimethylpropanoic acid S2-3A (130 mg, 90% purity, 0.317 mmol) in 4 M hydrochloride in ethyl acetate (4 mL) was stirred at 15° C. for 1 hour. The reaction mixture concentrated to give the title compound (80 mg, 90% purity from HNMR, 74.3% yield) as white solids. ¹H NMR (400 MHz, DMSO-d₆) δ 3.71-3.64 (m, 1H), 3.29-3.23 (m, 2H), 3.20-3.12 (m, 2H), 3.05-2.97 (m, 3H), 2.79 (d, J=12.4 Hz, 1H), 2.68-2.60 (m, 1H), 2.00-1.91 (m, 1H), 1.76-1.66 (m, 1H), 1.09 (s, 3H), 1.07 (s, 3H), 0.78 (t, J=7.2 Hz, 3H).

### Preparation of Intermediate S3-A

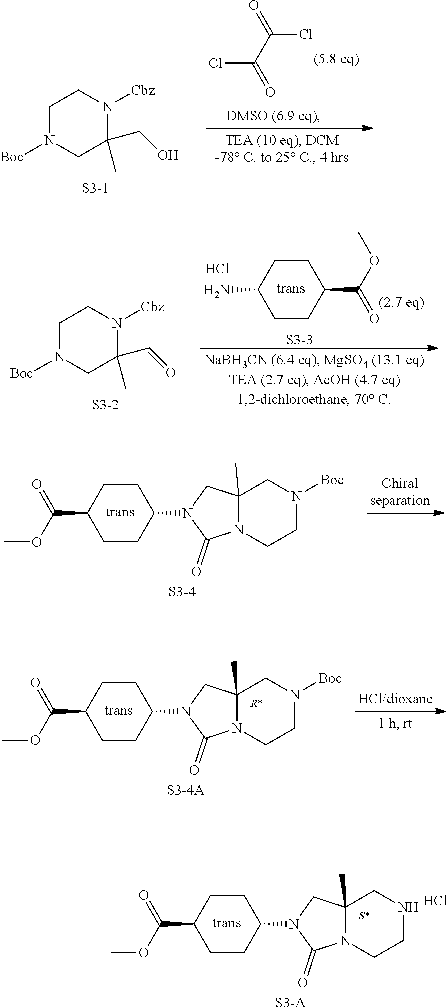

### Intermediate S3-2: 1-Benzyl 4-tert-butyl 2-formyl-2-methylpiperazine-1,4-dicarboxylate

To a solution of anhydrous dimethyl sulfoxide (1.20 g, 15.4 mmol) in anhydrous dichloromethane (50 mL) was added dropwise oxalyl dichloride (1.1 mL, 13.0 mmol) at −78° C. After stirred at −78° C. under nitrogen atmosphere for 1.5 hours, a solution of 1-benzyl 4-tert-butyl 2-(hydroxymethyl)-2-methylpiperazine-1,4-dicarboxylate S3-1 (900 mg, 90% purity, 2.22 mmol) in anhydrous dichloromethane (10 mL) was added dropwise. The mixture was stirred at −78° C. for 1.5 hours and triethylamine (3.1 mL, 22.2 mmol) was then added. The mixture was stirred at 25° C. for 1 hour. The reaction mixture was diluted with ice water (50 mL) and neutralized with 1 M hydrochloride aqueous solution to pH 6˜7. After that the mixture was extracted with dichloromethane (50 mL) for three times and the combined organic layers were washed with saturated sodium bicarbonate aqueous solution (50 mL) and brine (50 mL) for three times, dried over Na₂SO₄₍ₛ₎, filtered and evaporated to give the title compound (694 mg, 90% purity from HNMR, 78% yield) as light yellow oil. LC-MS (ESI): R_{T}=1.73 min, mass calcd. for C₁₉H₂₆N₂O₅ 362.2, m/z found 363.0 [M+H]⁺. ¹H NMR (400 MHz, DMSO-d₆) δ 9.36 (s, 1H), 7.38-7.33 (m, 5H), 5.14-5.06 (m, 2H), 3.88-3.84 (m, 1H), 3.76-3.63 (m, 1H), 3.48-3.06 (m, 4H), 1.39 (s, 9H), 1.24 (s, 3H).

### Intermediate S3-4: (trans)-tert-Butyl 2-(4-(methoxycarbonyl)cyclohexyl)-8a-methyl-3-oxohexahydroimidazo[1,5-a]pyrazine-7(1H)-carboxylate

To a solution of 1-benzyl 4-tert-butyl 2-formyl-2-methylpiperazine-1,4-dicarboxylate S3-2 (637 mg, 90% purity, 1.58 mmol) in 1,2-dichloroethane (20 mL) was added (trans)-methyl 4-aminocyclohexanecarboxylate hydrochloride S3-3 (818 mg, 4.22 mmol) and triethylamine (0.6 mL, 4.31 mmol) at room temperature. After stirred for 20 minutes with nitrogen protection, acetic acid (447 mg, 7.44 mmol) and magnesium sulphate (2.5 g, 20.8 mmol) was added and the reaction mixture was stirred at 70° C. overnight. Then sodium cyanoborohydride (637 mg, 10.1 mmol) was added and the mixture was stirring continued at 70° C. overnight. The reaction mixture was diluted with dichloromethane (30 mL) and water (80 mL), basified with sodium bicarbonate aqueous solution (about 10 mL) till pH to 8. The aqueous layer was extracted with dichloromethane (30 mL) for three times. The combined organic layers were washed with brine (30 mL), dried over Na₂SO₄₍ₛ₎ and filtered. The filtrate was concentrated under reduced pressure to give a residue, which was purified by C18 column (acetonitrile:water water (+0.02% ammonium acetate)=20% to 95%) to give the title compound (383 mg, 96% purity, 59% yield) as white solids. LC-MS (ESI): R_{T}=1.58 min, mass calcd. for C₂₀H₃₃N₃O₅ 395.2, m/z found 340.1 [M+H−56]⁺. ¹H NMR (400 MHz, DMSO-d₆) δ 3.88-3.65 (m, 2H), 3.59 (s, 3H), 3.51 (d, J=3.2 Hz, 1H), 3.47 (d, J=2.8 Hz, 1H), 3.09 (d, J=8.8 Hz, 1H), 3.02 (d, J=8.8 Hz, 1H), 2.85 (td, J=13.2, 3.6 Hz, 1H), 2.29-2.17 (m, 1H), 2.00-1.89 (m, 2H), 1.69-1.57 (m, 2H), 1.50-1.32 (m, 15H), 1.21 (s, 3H).

### Intermediates S3-4A and S3-4B: (trans)-(R*)-tert-Butyl 2-(4-(methoxycarbonyl)cyclohexyl)-8a-methyl-3-oxohexahydroimidazo[1,5-a]pyrazine-7(1H)-carboxylate and (trans)-(S*)-tert-butyl 2-(4-(methoxycarbonyl)cyclohexyl)-8a-methyl-3-oxohexahydroimidazo[1,5-a]pyrazine-7(1H)-carboxylate

A racemic mixture of (trans)-tert-butyl 2-(4-(methoxycarbonyl)cyclohexyl)-8a-methyl-3-oxohexahydroimidazo[1,5-a]pyrazine-7(1H)-carboxylate S3-4 (300 mg, 96% purity, 0.728 mmol) was separated by chiral Prep. HPLC (Column: Chiralpak IF 5 μm 20*250 mm, Mobile Phase:MeOH:EtOH=50:50 at 13 mL/min, Temp: 30° C., Wavelength: 214 nm) to afford the title compounds S3-4A (107 mg, 95% purity from NMR, 35% yield, 100% stereopure) and S3-4B (80 mg, 95% purity from NMR, 26% yield, 99.8% stereopure) as yellow solids.

Compound S3-4A: LC-MS (ESI): R_{T}=1.58 min, mass calcd. for C₂₀H₃₃N₃O₅ 395.2, m/z found 396.2 [M+H]⁺. Chiral analysis (Column: Chiralpak IF 5 μm 4.6*250 mm; Mobile Phase:MeOH:EtOH=50:50 at 1 mL/min; Temp: 30° C.; Wavelength: 214 nm, R_{T}=8.887 min). ¹H NMR (400 MHz, DMSO-d₆) δ 3.87-3.64 (m, 2H), 3.59 (s, 3H), 3.51 (d, J=3.2 Hz, 1H), 3.48-3.47 (m, 1H), 3.08 (d, J=9.2 Hz, 1H), 3.02 (d, J=9.2 Hz, 1H), 2.85 (td, J=12.8, 4.0 Hz, 1H), 2.27-2.18 (m, 1H), 2.01-1.88 (m, 2H), 1.69-1.56 (m, 2H), 1.47-1.43 (m, 3H), 1.40-1.33 (m, 12H), 1.21 (s, 3H).

Compound S3-4B: LC-MS (ESI): R_{T}=1.58 min, mass calcd. for C₂₀H₃₃N₃O₅ 395.2, m/z found 340.1 [M+H-56]⁺. Chiral analysis (Column: Chiralpak IF 5 μm 4.6*250 mm; Mobile Phase:MeOH:EtOH=50:50 at 1 mL/min; Temp: 30° C.; Wavelength: 214 nm, R_{T}=11.261 min). ¹H NMR (400 MHz, DMSO-d₆) δ 3.87-3.64 (m, 2H), 3.59 (s, 3H), 3.51 (d, J=3.2 Hz, 1H), 3.48-3.47 (m, 1H), 3.08 (d, J=9.2 Hz, 1H), 3.02 (d, J=9.2 Hz, 1H), 2.85 (td, J=12.4, 3.6 Hz, 1H), 2.28-2.19 (m, 1H), 2.01-1.90 (m, 2H), 1.68-1.57 (m, 2H), 1.49-1.44 (m, 3H), 1.40-1.34 (m, 12H), 1.21 (s, 3H).

### Intermediate S3-A: (trans)-Methyl 4-((S)-8a-methyl-3-oxohexahydroimidazo[1,5-a]pyrazin-2(3H)-yl)cyclohexanecarboxylate Hydrochloride

A solution of (trans)-(R*)-tert-butyl 2-(4-(methoxycarbonyl)cyclohexyl)-8a-methyl-3-oxohexahydroimidazo[1,5-a]pyrazine-7(1H)-carboxylate S3-4A (107 mg, 95% purity, 0.257 mmol) in 6 M hydrochloride(g) in 1,4-dioxane solution (10 mL) was stirred for 1 hour at room temperature, then the mixture was concentrated under reduced pressure to afford the compound (86 mg, 94% purity, 95% yield) as yellow solids. LC-MS (ESI): R_{T}=0.219 min, mass calcd. for C₁₅H₂₆ClN₃O₃ 331.2, m/z found 296.2 [M+H−HCl]⁺.

### Preparation of Intermediate S4-A

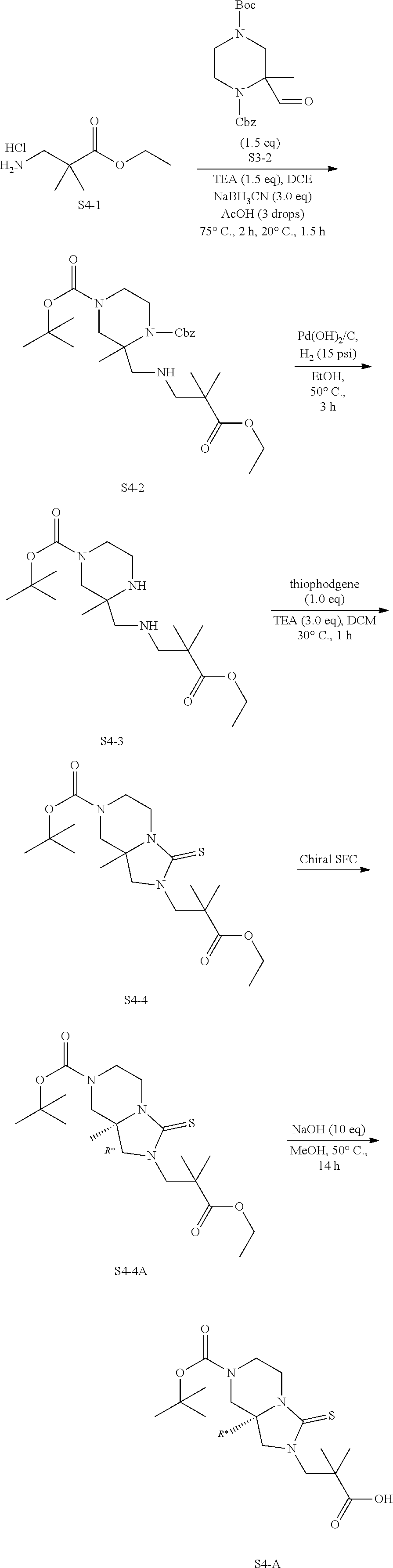

### Intermediate S4-2: Benzyl 4-tert-butyl 2-(((3-ethoxy-2,2-dimethyl-3-oxopropyl)amino)methyl)-2-methylpiperazine-1,4-dicarboxylate

To a solution of ethyl 3-amino-2,2-dimethylpropanoate hydrochloride S4-1 (500 mg, 1.38 mmol) in 1,2-dichloroethane (5 mL) was added triethylamine (209 mg, 2.07 mmol). After stirred at 20° C. for 10 minutes, 1-benzyl 4-tert-butyl 2-formyl-2-methylpiperazine-1,4-dicarboxylate S3-2 (375 mg, 2.07 mmol) and acetic acid (three drops) were added into the mixture and it was stirred at 75° C. for 2 hours, then sodium cyanoborohydride (260 mg, 4.14 mmol) was added. The resulting mixture was stirred at 20° C. for 1.5 hours, filtered and concentrated under reduced pressure to get a residue, which was purified by C18 column (acetonitrile:water=5% to 100%) to give the title compound (400 mg, 90% purity from ¹H NMR, 59% yield) as yellow oil. ¹H NMR (400 MHz, CDCl₃) δ 7.38-7.32 (m, 5H), 5.11 (s, 2H), 4.09 (q, J=7.2 Hz, 2H), 3.84 (br s, 1H), 3.77-3.73 (m, 1H), 3.63-3.56 (m, 1H), 3.42-3.15 (m, 4H), 2.67-2.59 (m, 3H), 1.46 (s, 9H), 1.31 (s, 3H), 1.22 (t, J=7.2 Hz, 3H), 1.13-1.12 (m, 6H).

### Intermediate S4-3: tert-Butyl 3-(((3-ethoxy-2,2-dimethyl-3-oxopropyl)amino)methyl)-3-methylpiperazine-1-carboxylate

To a solution of benzyl 4-tert-butyl 2-(((3-ethoxy-2,2-dimethyl-3-oxopropyl)amino)methyl)-2-methylpiperazine-1,4-dicarboxylate S4-2 (400 mg, 90% purity, 0.810 mmol) in ethanol (10 mL) was added 10% palladium hydroxide on activated carbon wt. (200 mg). After stirred at 50° C. for 3 hours under hydrogen atmosphere (15 psi), the reaction mixture was filtered and concentrated to afford the title compound (260 mg, 90% purity from ¹HNMR, 90% yield) as yellow oil. ¹H NMR (400 MHz, CDCl₃) δ 4.10 (q, J=7.2 Hz, 2H), 3.36-3.30 (m, 2H), 3.22-3.11 (m, 2H), 2.79-2.74 (m, 3H), 2.64-2.59 (m, 2H), 2.47-2.44 (m, 1H), 1.44 (s, 9H), 1.23 (t, J=7.2 Hz, 3H), 1.13-1.16 (m, 6H), 1.00 (s, 3H).

### Intermediate S4-4: tert-Butyl 2-(3-ethoxy-2,2-dimethyl-3-oxopropyl)-8a-methyl-3-thioxohexahydroimidazo[1,5-a]pyrazine-7(1H)-carboxylate

A solution of triphosgene (126 mg, 0.730 mmol) in dichloromethane (1 mL) was added into a mixture of tert-butyl 3-(((3-ethoxy-2,2-dimethyl-3-oxopropyl)amino)methyl)-3-methylpiperazine-1-carboxylate S4-3 (260 mg, 90% purity, 0.730 mmol) and triethylamine (221 mg, 2.19 mmol) in dichloromethane (5 mL). The mixture was stirred at 30° C. for 1 hour. The mixture was diluted with water (30 mL), extracted with dichloromethane (30 mL) twice, the combined organic layers were washed with water (100 mL), dried over Na₂SO₄₍ₛ₎, fittered and concentrated under reduced pressure to get a residue, which was purified by silica gel column chromatography (petroleum ether:ethyl acetate=8:1) to give the title compound (160 mg, 90% purity from ¹H NMR, 55% yield) as brown oil. ¹H NMR (400 MHz, CDCl₃) δ 4.44-4.41 (m, 1H), 4.16 (q, J=7.2 Hz, 2H), 4.01-3.79 (m, 4H), 3.31-3.16 (m, 3H), 2.91-2.70 (m, 2H), 1.47 (s, 9H), 1.33 (s, 3H), 1.28-1.25 (m, 9H).

### Intermediates S4-4A and S4-4B (R*)-tert-Butyl 2-(3-ethoxy-2,2-dimethyl-3-oxopropyl)-8a-methyl-3-thioxohexahydroimidazo[1,5-a]pyrazine-7(1H)-carboxylate and (S*)-tert-butyl 2-(3-ethoxy-2,2-dimethyl-3-oxopropyl)-8a-methyl-3-thioxohexahydroimidazo[1,5-a]pyrazine-7(1H)-carboxylate

A racemic mixture of (tert-butyl 2-(3-ethoxy-2,2-dimethyl-3-oxopropyl)-8a-methyl-3-thioxohexahydroimidazo[1,5-a]pyrazine-7(1H)-carboxylate S4-4 (140 mg, 0.350 mmol) was separated by chiral Prep. SFC (Column: Chiralpak IG 5 μm 20*250 mm; Mobile Phase: CO₂: MeOH=80:20 at 50 g/min; Col. Temp: 30° C.; Wavelength: 230 nm) to afford the title compounds S4-4A (60 mg, 90% purity from HNMR, 43% yield, 100% ee) and S4-4B (50 mg, 90% purity from HNMR, 36% yield, 99.2% ee) as yellow oil.

S4-4A: Chiral analysis (Column: Chiralpak IG 5 μm 4.6*250 mm; Mobile Phase: CO₂: MeOH=80:20 at 3.0 g/min; Col. Temp: 40° C.; Wavelength: 230 nm, Back pressure: 100 bar, R_{T}=2.25 min). ¹H NMR (400 MHz, CDCl₃) δ 4.41-4.38 (m, 1H), 4.14 (t, J=6.8 Hz, 2H), 3.98-3.83 (m, 4H), 3.29-3.16 (m, 3H), 2.81-2.67 (m, 2H), 1.45 (s, 9H), 1.28-1.23 (m, 12H).

S4-4B: Chiral analysis (Column: Chiralpak IG 5 μm 4.6*250 mm; Mobile Phase: CO₂: MeOH=80:20 at 3.0 g/min; Col. Temp: 40° C.; Wavelength: 230 nm, Back pressure: 100 bar, R_{T}=3.01 min). ¹H NMR (400 MHz, CDCl₃) δ 4.42-4.39 (m, 1H), 4.14 (t, J=7.2 Hz, 2H), 4.00-3.75 (m, 4H), 3.40-3.20 (m, 3H), 2.88-2.68 (m, 2H), 1.45 (s, 9H), 1.33-1.24 (m, 12H).

### Intermediate S4-A: (R*)-3-(7-(tert-Butoxycarbonyl)-8a-methyl-3-thioxohexahydroimidazo[1,5-a]pyrazin-2(3H)-yl)-2,2-dimethylpropanoic Acid

To a solution of sodium hydroxide (60 mg, 1.50 mmol) in water (1 mL) was added a mixture of (R*)-tert-butyl 2-(3-ethoxy-2,2-dimethyl-3-oxopropyl)-8a-methyl-3-thioxohexahydroimidazo[1,5-a]pyrazine-7(1H)-carboxylate S4-4A (60 mg, 90% purity, 0.150 mmol) in methanol (3 mL). After stirred at 50° C. for 14 hours, the reaction mixture was diluted with water (10 mL), removed methanol under vacuo. The residue was acidified with 1 M hydrochloride aqueous solution to pH 5˜6, extracted with ethyl acetate (10 mL) for three times. The combined organic layers were washed with brine (10 mL), dried over Na₂SO₄₍ₛ₎ and filtered. The filtrate was concentrated to give the title compound (45 mg, 97% purity, 97% yield) as yellow oil. LC-MS (ESI): R_{T}=1.290 min, mass calcd. for C₁₇H₂₉N₃O₄S, 371.2, m/z found 372.2.

### Compound 1: 3-(7-(((S)-5-(ethoxycarbonyl)-6-(3-fluoro-2-methylphenyl)-2-(thiazol-2-yl)-3,6-dihydropyrimidin-4-yl)methyl)-8a-methyl-3-oxohexahydroimidazo[1,5-a]pyrazin-2(3H)-yl)-2,2-dimethylpropanoic Acid

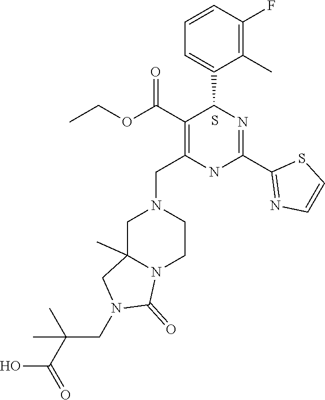

### Compound 1

### 3-(7-(((S)-5-(Ethoxycarbonyl)-6-(3-fluoro-2-methylphenyl)-2-(thiazol-2-yl)-3,6-dihydropyrimidin-4-yl)methyl)-8a-methyl-3-oxohexahydroimidazo[1,5-a]pyrazin-2(3H)-yl)-2,2-dimethylpropanoic Acid (Mixture of 2 Diastereomers)

To a solution of 2,2-dimethyl-3-(8a-methyl-3-oxohexahydroimidazo[1,5-a]pyrazin-2(3H)-yl)propanoic acid hydrochloride S1 (120 mg, 0.371 mmol) in dichloromethane (3 mL) was added triethanolamine (276 mg, 1.85 mmol). After stirred at 40° C. for 30 minutes, a solution of (S)-ethyl 6-(bromomethyl)-4-(3-fluoro-2-methylphenyl)-2-(thiazol-2-yl)-1,4-dihydropyrimidine-5-carboxylate H2-1A (170 mg, 95% purity, 0.371 mmol) in dichloromethane (2 mL) was added dropwise. After stirred at 40° C. for 16 hours, the reaction mixture was concentrated to give a residue, which was purified by Prep. HPLC (Column: waters Xbridge C18 (5 μm 19*150 mm), Mobile Phase A: water (0.1% trifluoroacetic acid), Mobile Phase B: acetonitrile, UV: 214 nm, Flow rate: 15 mL/min, Gradient: 15-60% (% B)) and further purified by C18 (acetonitrile:water (0.1% ammonium bicarbonate)=20% to 50%) give the title compound (59.8 mg, 26% yield) as yellow solids. LC-MS (ESI): R_{T}=3.208 min, mass calcd. for C₃₀H₃₇FN₆O₅S 612.3, m/z found 613.2[M+H]⁺. ¹H NMR (400 MHz, DMSO-d₆) δ 8.00 (d, J=3.2 Hz, 0.5H), 7.96 (d, J=2.8 Hz, 0.5H), 7.93 (d, J=3.2 Hz, 0.5H), 7.92 (d, J=3.2 Hz, 0.5H), 7.21-7.15 (m, 1H), 7.05-7.01 (m, 2H), 5.89 (s, 0.5H), 5.88 (s, 0.5H), 4.00-3.88 (m, 3H), 3.80 (dd, J=16.4, 4.0 Hz, 1H), 3.63-3.55 (m, 1H), 3.30 (dd, J=14.0, 4.0 Hz, 1H), 3.18-3.00 (m, 4H), 2.80-2.56 (m, 2H), 2.45 (s, 3H), 2.29 (d, J=10.8 Hz, 0.5H), 2.14-1.94 (m, 1.5H), 1.51 (s, 1.5H), 1.39 (s, 1.5H), 1.07-1.02 (m, 9H).

### Compound 1B

### 3-(7-(((S)-5-(Ethoxycarbonyl)-6-(3-fluoro-2-methylphenyl)-2-(thiazol-2-yl)-3,6-dihydropyrimidin-4-yl)methyl)-8a-methyl-3-oxohexahydroimidazo[1,5-a]pyrazin-2(3H)-yl)-2,2-dimethylpropanoic Acid (A Single Enantiomer)

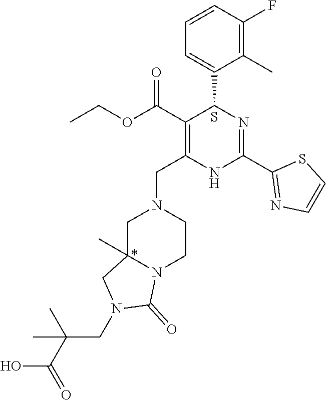

This compound was prepared from S1-A and H2-1A using same condition as for compound 1. LC-MS (ESI): R_{T}=3.556 min, mass calcd. for C₃₀H₃₇FN₆O₅S 612.3, m/z found 613.3 [M+H]⁺. ¹H NMR (400 MHz, DMSO-d₆) δ 9.55 (br s, 1H), 7.97 (d, J=2.8 Hz, 1H), 7.92 (d, J=2.8 Hz, 1H), 7.21-7.15 (m, 1H), 7.06-7.01 (m, 2H), 5.88 (s, 0.9H), 5.75 (s, 0.1H), 3.97 (q, J=7.2 Hz, 2H), 3.91 (d, J=16.8 Hz, 1H), 3.81 (d, J=16.8 Hz, 1H), 3.62 (d, J=11.6 Hz, 1H), 3.31 (d, J=13.6 Hz, 1H), 3.14-3.01 (m, 4H), 2.80 (d, J=9.6 Hz, 1H), 2.59 (d, J=11.2 Hz, 1H), 2.45 (s, 1.5H), 2.44 (s, 1.5H), 2.14-2.04 (m, 2H), 1.40 (s, 3H), 1.08-1.02 (m, 9H).

### Compound 2A

### (S*)-3-(-7-(((R*)-6-(2-Chloro-3-fluorophenyl)-5-(ethoxycarbonyl)-2-(thiazol-2-yl)-3,6-dihydropyrimidin-4-yl)methyl)-8a-methyl-3-oxohexahydroimidazo[1,5-a]pyrazin-2(3H)-yl)-2,2-dimethylpropanoic Acid (A Single Enantiomer)

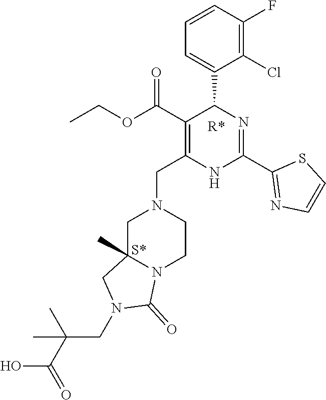

A suspension of S1-A (61 mg, 90% purity, 0.188 mmol) in dichloromethane (3 mL) was added triethanolamine (117 mg, 0.784 mmol). After the mixture was warmed up to 35° C., H1-1A (80 mg, 90% purity, 0.157 mmol) in dichloromethane (2 mL) were added at 35° C. Then the mixture was stirred at 35° C. for 16 hours. The reaction was quenched with water (5 mL) and acidified pH with 0.5 M hydrochloride aqueous solution from 5 to 6. The aqueous phase was extracted with dichloromethane (10 mL) for thress times, then the combined organic phases were dried over Na₂SO₄₍ₛ₎, filtered and the filtrate was concentrated to afford the crude product, which was purified by C18 column (acetonitrile:water (0.1% ammonium bicarbonate)=35% to 90%) to afford the title product (92.0 mg, 99% purity, 92% yield) as yellow solids. LC-MS (ESI): R_{T}=3.572 min, mass calcd. for C₂₉H₃₄ClFN₆O₅S 632.2, m/z found 633.2 [M+H]⁺. ¹H NMR (400 MHz, CD₃OD) δ 7.90 (d, J=3.2 Hz, 1H), 7.72 (d, J=3.2 Hz, 1H), 7.31-7.22 (m, 2H), 7.13 (dt, J=8.8, 1.6 Hz, 1H), 6.21 (s, 1H), 4.05-3.99 (m, 3H), 3.82 (d, J=17.2 Hz, 1H), 3.76-3.72 (m, 1H), 3.42 (d, J=14.0 Hz, 1H), 3.29-3.14 (m, 4H), 2.87-2.84 (m, 1H), 2.62 (d, J=11.2 Hz, 1H), 2.30-2.21 (m, 2H), 1.53 (s, 3H), 1.19 (s, 3H), 1.17 (s, 3H), 1.09 (t, J=7.2 Hz, 3H).

### Compound 3A

### 3-((S*)-7-(((R*)-6-(2-Bromo-4-fluorophenyl)-5-(methoxycarbonyl)-2-(thiazol-2-yl)-3,6-dihydropyrimidin-4-yl)methyl)-8a-methyl-3-oxohexahydroimidazo[1,5-a]pyrazin-2(3H)-yl)-2,2-dimethylpropanoic Acid

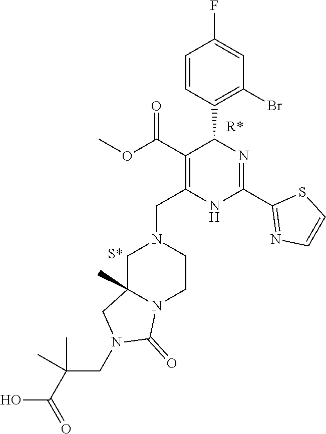

To the solution of S1-A (100 mg, 90% purity, 0.308 mmol) in dichloromethane (20 mL) was added triethanolamine (368 mg, 2.47 mmol). The mixture was stirred at room temperature for 10 minutes before H3-1A (220 mg, 90% purity, 0.405 mmol) was added. After stirred at 40° C. under nitrogen atmosphere for 2.5 hours and then stirred at room temperature overnight. The mixture was diluted with water (50 mL), which was extracted with dichloromethane (50 mL) twice. The combined organic layers were dried over Na₂SO₄₍ₛ₎, filtered and concentrated. The residue was purified by C18 column (acetonitrile:water (0.1% ammonium bicarbonate)=05% to 70%) to afford the title compound (90.2 mg, 99.7% purity, 44% yield) as yellow solids. LC-MS (ESI): R_{T}=3.285 min, mass calcd. for C₂₈H₃₂BrFN₆O₅S 662.1, m/z found 663.2 [M+H]⁺. ¹H NMR (400 MHz, DMSO-d₆) δ 9.82-9.29 (m, 1H), 7.98 (d, J=3.2 Hz, 1H), 7.93 (d, J=3.2 Hz, 1H), 7.56 (dd, J=8.4 Hz, 2.4 Hz, 1H), 7.40-7.36 (m, 1H), 7.24-7.19 (m, 1H), 6.01 (s, 1H), 3.88 (d, J=16.8 Hz, 1H), 3.80 (d, J=17.2 Hz, 1H), 3.63-3.60 (m, 1H), 3.51 (s, 3H), 3.30 (d, J=14 Hz, 1H), 3.13-3.07 (m, 3H), 3.05-3.01 (m, 1H), 2.81-2.79 (m, 1H), 2.59 (d, J=10.8 Hz, 1H), 2.16-2.05 (m, 2H), 1.41 (s, 3H), 1.07 (s, 3H), 1.06 (s, 3H).

### Compound 4B

### 3-((S*)-7-(((S)-5-(Ethoxycarbonyl)-6-(3-fluoro-2-methylphenyl)-2-(thiazol-2-yl)-3,6-dihydropyrimidin-4-yl)methyl)-8a-ethyl-3-oxohexahydroimidazo[1,5-a]pyrazin-2(3H)-yl)-2,2-dimethylpropanoic Acid

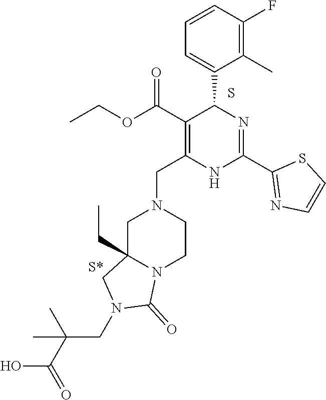

To a solution of (S*)-3-(8a-ethyl-3-oxohexahydroimidazo[1,5-a]pyrazin-2(3H)-yl)-2,2-dimethylpropanoic acid hydrochloride S2-A (80 mg, 90% purity, 0.235 mmol) in dichloromethane (3 mL) was added triethanolamine (176 mg, 1.18 mmol). After stirred at 40° C. for 30 minutes, a solution of (S)-ethyl 6-(bromomethyl)-4-(3-fluoro-2-methylphenyl)-2-(thiazol-2-yl)-1,4-dihydropyrimidine-5-carboxylate H2-1A (116 mg, 90% purity, 0.238 mmol) in dichloromethane (2 mL) was added dropwise. After stirred at 40° C. for 16 hours, the reaction mixture was concentrated to give a residue, which was purified by prep-HPLC (Column: Waters Xbridge C18 (5 μm 19*150 mm), Mobile Phase A: Water (0.1% ammonium bicarbonate), Mobile Phase B: acetonitrile, UV: 214 nm, Flow rate: 15 mL/min, Gradient: 05-70% (% B)) to give the title compound (75.6 mg, 99.0% purity, 50.7% yield) as yellow solids. LC-MS (ESI): R_{T}=3.489 min, mass calcd. for C₃₁H₃₉FN₆O₅S 626.3, m/z found 627.2[M+H]⁺. ¹H NMR (400 MHz, DMSO-d₆) δ 9.52 (br s, 1H), 7.98 (d, J=3.2 Hz, 1H), 7.93 (d, J=3.2 Hz, 1H), 7.24-7.15 (m, 1H), 7.06-7.01 (m, 2H), 5.88 (s, 0.9H), 5.75 (s, 0.1H), 4.00-3.95 (m, 3H), 3.77 (d, J=16.4 Hz, 1H), 3.64 (d, J=12.0 Hz, 1H), 3.29 (d, J=13.6 Hz, 1H), 3.15 (d, J=13.6 Hz, 1H), 3.08 (d, J=9.2 Hz, 1H), 3.03-2.91 (m, 2H), 2.80 (d, J=10.0 Hz, 1H), 2.61 (d, J=11.6 Hz, 1H), 2.45 (s, 3H), 2.13-2.04 (m, 2H), 1.96-1.89 (m, 1H), 1.78-1.69 (m, 1H), 1.07-1.03 (m, 9H), 0.60 (t, J=7.2 Hz, 3H).

### Compound 5A

### (trans)-4-((S)-7-(((S*)-5-(Ethoxycarbonyl)-6-(3-fluoro-2-methylphenyl)-2-(thiazol-2-yl)-3,6-dihydropyrimidin-4-yl)methyl)-8a-methyl-3-oxohexahydroimidazo[1,5-a]pyrazin-2(3H)-yl)cyclohexanecarboxylic Acid

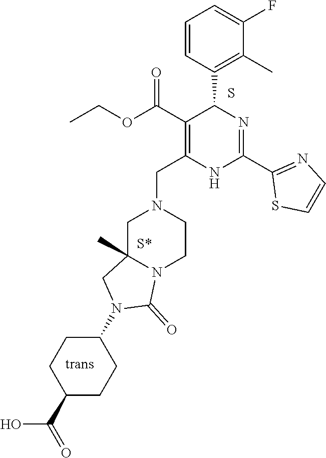

### Intermediate 5A-1

### (trans)-(S)-Ethyl 4-(3-fluoro-2-methylphenyl)-6-(((S*)-2-(4-(methoxycarbonyl)cyclohexyl)-8a-methyl-3-oxohexahydroimidazo[1,5-a]pyrazin-7(1H)-yl)methyl)-2-(thiazol-2-yl)-1,4-dihydropyrimidine-5-carboxylate

To a solution of (trans)-methyl 4-((S*)-8a-methyl-3-oxohexahydroimidazo[1,5-a]pyrazin-2(3H)-yl)cyclohexanecarboxylate hydrochloride S3-A (75 mg, 94% purity, 0.21 mmol) in dichloromethane (10 mL) was added triethanolamine (153 mg, 1.03 mmol) at room temperature. After stirring at 40° C. under nitrogen atmosphere for 15 minutes, (9-ethyl 6-(bromomethyl)-4-(3-fluoro-2-methylphenyl)-2-(thiazol-2-yl)-1,4-dihydropyrimidine-5-carboxylate H2-1A (100 mg, 90% purity, 0.205 mmol) was added at 40° C. After stirred at room temperature for 12 hours under nitrogen atmosphere, the mixture was diluted with dichloromethane (30 mL) and water (30 mL). The aqueous layer was extracted with dichloromethane (30 mL) for three times. The combined organic layers were washed with brine (30 mL), dried over Na₂SO₄₍ₛ₎ and filtered. The filtrate was concentrated under reduced pressure to give a residue, which was purified by C18 column (acetonitrile:water (0.1% ammonium bicarbonate)=20% to 95%) to give the title compound (122 mg, 95% purity from NMR, 86% yield) as yellow solids. LC-MS (ESI): R_{T}=1.826 min, mass calcd. for C₃₃H₄₁FN₆O₅S 652.2, m/z found 653.3 [M+H]⁺. ¹H NMR (400 MHz, DMSO-d₆) δ 9.54 (s, 1H), 7.98-7.93 (m, 2H), 7.21-7.15 (m, 1H), 7.05-7.03 (m, 2H), 5.87 (s, 1H), 4.11-3.95 (m, 3H), 3.94-3.77 (m, 1H), 3.66-3.57 (m, 4H), 3.54-3.46 (m, 1H), 3.07-2.95 (m, 3H), 2.79-2.64 (m, 2H), 2.44 (s, 3H), 2.24-2.11 (m, 2H), 2.08-1.95 (m, 3H), 1.66-1.56 (m, 2H), 1.51-1.31 (m, 7H), 1.06-1.02 (m, 3H).

### Compound 5A

### (trans)-4-((S)-7-(((S*)-5-(Ethoxycarbonyl)-6-(3-fluoro-2-methylphenyl)-2-(thiazol-2-yl)-3,6-dihydropyrimidin-4-yl)methyl)-8a-methyl-3-oxohexahydroimidazo[1,5-a]pyrazin-2(3H)-yl)cyclohexanecarboxylic Acid

To a solution of (trans)-(S)-ethyl 4-(3-fluoro-2-methylphenyl)-6-((S*)-2-(4-(methoxycarbonyl)cyclohexyl)-8a-methyl-3-oxohexahydroimidazo[1,5-a]pyrazin-7(1H)-yl)methyl)-2-(thiazol-2-yl)-1,4-dihydropyrimidine-5-carboxylate 5A-1 (122 mg, 95% purity, 0.178 mmol) in tetrahydrofuran (10 mL) and water (10 mL) was added lithium hydroxide monohydrate (80 mg, 1.9 mmol) at room temperature. After stirred at room temperature for 2 hours, the mixture was acidified with 1 M hydrochloride aqueous solution (about 3 mL) till pH to 1-2. The aqueous layer was extracted with ethyl acetate (30 mL) for three times. The combined organic layers were washed with brine (30 mL), dried over Na₂SO₄₍ₛ₎ and filtered. The filtrate was concentrated under reduced pressure to give a residue, which was purified by C18 column (acetonitrile:water (0.1% ammonium bicarbonate)=20% to 95%) to give the title compound (80 mg, 99.8% purity, 70% yield) as yellow solids. LC-MS (ESI): R_{T}=3.717 min, mass calcd. for C₃₂H₃₉FN₆O₅S 638.2, m/z found 639.3 [M+H]⁺. ¹H NMR (400 MHz, DMSO-d₆) δ 9.55 (br s, 1H), 7.98 (s, 0.1H), 7.96 (d, J=3.2 Hz, 1H), 7.91 (d, J=3.2 Hz, 0.9H), 7.20-7.14 (m, 1H), 7.04-7.00 (m, 2H), 5.89 (s, 1H), 3.95 (q, J=6.8 Hz, 2H), 3.90 (d, J=16.4 Hz, 1H), 3.78 (d, J=16.4 Hz, 1H), 3.62-3.59 (m, 1H), 3.53-3.46 (m, 1H), 3.06-2.99 (m, 2H), 2.95 (d, J=8.8 Hz, 1H), 2.76 (d, J=10.0 Hz, 1H), 2.54 (d, J=11.2 Hz, 1H), 2.43 (d, J=1.6 Hz, 3H), 2.11-2.03 (m, 3H), 1.91-1.85 (m, 2H), 1.67-1.52 (m, 2H), 1.42-1.30 (m, 7H), 1.03 (t, J=6.8 Hz, 3H).

### Compound 6A

### 3-((R*)-7-(((S)-5-(Ethoxycarbonyl)-6-(3-fluoro-2-methylphenyl)-2-(thiazol-2-yl)-3,6-dihydropyrimidin-4-yl)methyl)-8a-methyl-3-thioxohexahydroimidazo[1,5-a]pyrazin-2(3H)-yl)-2,2-dimethylpropanoic Acid

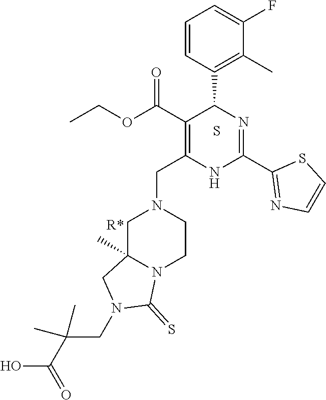

(R*)-3-(7-(tert-butoxycarbonyl)-8a-methyl-3-thioxohexahydroimidazo[1,5-a]pyrazin-2(3H)-yl)-2,2-dimethylpropanoic acid S4-A (45 mg, 97% purity, 0.12 mmol) in 4 M hydrochloride in ethyl acetate (4 mL, 16 mmol) was stirred at 20° C. for 1 hour. The mixture was concentrated to give a residue, which was diluted in tetrahydrofuran (5 mL), then triethylamine (39 mg, 0.38 mmol) was added. After stirred at 20° C. for 0.5 hour, (S)-ethyl6-(bromomethyl)-4-(3-fluoro-2-methylphenyl)-2-(thiazol-2-yl)-1,4-dihydropyrimidine-5-carboxylate H2-1A (43 mg, 95% purity, 0.098 mmol) was added to the mixture. After stirred at 40° C. for 3 hours, the mixture was stirred at 20° C. for 14 hours, was and then diluted with 0.01 M hydrochloride aqueous solution (20 mL) and extracted with ethyl acetate (20 mL) twice. The combined organic layers were washed with brine (20 mL), dried over Na₂SO₄₍ₛ₎ and filtered and concentrated. The residue was purified by C18 column (acetonitrile:water (0.1% ammonium bicarbonate)=45% to 50%) to give the title compound (9.3 mg, 99.3% purity, 15% yield) as yellow solids. LC-MS (ESI): R_{T}=3.810 min, mass calcd. for C₃₀H₃₇FN₆O₄S₂ 628.2, m/z found 629.3. ¹H NMR (400 MHz, CD₃OD) δ 7.91 (d, J=3.2 Hz, 1H), 7.72 (d, J=3.2 Hz, 1H), 7.19-7.10 (m, 2H), 6.96-6.92 (m, 1H), 5.98 (s, 1H), 4.49-4.45 (m, 1H), 4.09-4.03 (m, 3H), 3.93-3.82 (m, 3H), 3.51-3.39 (m, 3H), 2.95-2.92 (m, 1H), 2.74-2.71 (m, 1H), 2.52 (s, 3H), 2.42-2.38 (m, 1H), 2.36-2.27 (m, 1H), 1.59 (s, 3H), 1.19-1.12 (m, 9H).

### Example 1: Anti-Viral Assay in HepG2.2.15 Cells

### Materials and Equipments

1) Cell Line

HepG2.2.15 (the HepG2.2.15 cell line can be produced by transfection of the HepG2 cell line as described in Sells, Chen, and Acs 1987 (Proc. Natl. Acad. Sci. USA 84:1005-1009), and the HepG2 cell line is available from ATCC® under number HB-8065™).

2) Reagents
DMEM/F12 (INVITROGEN-11330032)
FBS (GIBCO-10099-141)
Dimethyl sulfoxide (DMSO) (SIGMA-D2650)
Penicillin-streptomycin solution (HYCLONE-SV30010)
NEAA (INVITROGEN-1114050)
L-Glutamine (INVITROGEN-25030081)
Geneticin Selective Antibiotic (G418, 500 mg/ml) (INVITROGEN-10131027)
Trypsinase digestion solution (INVITROGEN-25300062)
CCK8 (BIOLOTE-35004)
QIAamp 96 DNA Blood Kit (12) (QIAGEN-51162)
FastStart Universal Probe Mast Mix (ROCHE-04914058001)

3) Consumables
96-well cell culture plate (COSTAR-3599)
Micro Amp Optical 96-well reaction plate (APPLIED BIOSYSTEMS-4306737)
Micro Amp Optical 384-well reaction plate (APPLIED BIOSYSTEMS)

4) Equipment
Plate reader (MOLECULAR DEVICES, SPECTRAMAX M2e)
Centrifuge (BECKMAN, ALLEGRA-X15R)
Real Time PCR system (APPLIED BIOSYSTEMS, QUANTSTUDIO 6)
Real Time PCR system (APPLIED BIOSYSTEMS, 7900HT)

### Methods

1) Anti-HBV Activity and Cytotoxicity Determination

HepG2.2.15 cells were plated into 96-well plate in 2% FBS culture medium at the density of 40,000 cells/well and 5,000 cells/well for HBV inhibitory activity and cytotoxicity determination, respectively. After incubation at 37° C., 5% CO2 overnight, cells were treated with medium containing compounds for 6 days with medium and compounds refreshed after 3 days of treatment. Each compound was tested in a 1:3 serial dilutions at 8 different concentrations in triplicate. The highest concentration of the compounds was 10 uM or 1 uM for anti-HBV activity assay and 100 uM for cytotoxicity determination.

Cell viability was determined by CCK-8 assay. After 6 days of compounds treatment, 20 μl CCK-8 reagents were added to each well of cytotoxicity assay plates. Cell plates were incubated at 37° C., 5% CO2 for 2.5 h. The absorbance at 450 nm wavelength and the absorbance at 630 nm wavelength as reference was measured.

The change of HBV DNA level induced by the compounds was assessed by quantitative real-time polymerase chain reaction (qPCR). Briefly, the HBV DNA in the culture medium was extracted using QIAamp 96 DNA Blood Kit according to the manual and then quantified by real-time PCR assay using the primers and probe in the table 1 below.

TABLE 1

| Primers | | |
|---|---|---|
| or Probe | Sequence | SEQ ID NO: |
| HBV-Fw | GTGTCTGCGGCGTTTTATCA | 1 |
| HBV-Rev | GACAAACGGGCAACATACCTT | 2 |
| HBV-Probe | CCTCTKCATCCTGCTGCTA | 3 |
| With FAM | TGCCTCATC | |
| reporter | | |
| and TAMRA | | |
| quencher | | |

2) DATA Analysis

EC50 and CC50 values are calculated by the GRAPHPAD PRISM software. If the CV % of DMSO controls is below 15% and the reference compounds shows expected activity or cytotoxicity, the data of this batch of experiment is considered qualified.

RESULTS: See Table 2 below.

TABLE 2

| Compound | EC₅₀ | CC₅₀ |
|---|---|---|
| ID | (uM) | (uM) |
| 1B | 0.009 | 24.3 |
| 2A | 0.016 | 19.4 |
| 3A | 0.038 | 26.7 |
| 4B | 0.165 | 18.5 |
| 5A | 0.034 | 9.2 |
| 6A | 0.012 | 13 |

1. A compound of Formula (I) 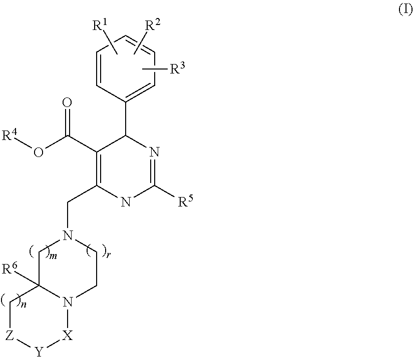
including the deuterated isomers, stereoisomers or tautomeric forms thereof, or a pharmaceutically acceptable salt thereof, wherein:
R¹, R² and R³ are independently selected from the group consisting of H, halogen and C₁₋₄ alkyl;
R⁴ is C₁₋₄alkyl;
R⁵ is thiazolyl, or pyridyl optionally substituted with one or more substituents selected from the group consisting of fluorine and C₁₋₃ Alkyl;
R⁶ is C₁₋₄alkyl, optionally substituted with a substituent selected from the group consisting of OH and CN;
m is 1;
r is 1;
n is an integer of 0 or 1;
X is C(═O), C(═S), or SO₂;
Y is NR⁷;
R⁷ is selected from the group consisting of H, —C₁₋₆alkyl, —C₁₋₆alkyl-R⁸, —C₁₋₆alkoxy-C₁₋₆alkyl-R⁸, —(CH₂)ₚ—C(R¹¹R¹²)—R⁸ and —(CH₂)ₚ-Q-R⁸;
R⁸ is selected from the group consisting of —C₁₋₆alkyl, —COOH, —C(═O)NHS(═O)₂—C₁₋₆alkyl, tetrazolyl, and carboxylic acid bioisosteres;
R¹¹ and R¹² together with carbon atom to which they are attached form a 3-7 membered saturated ring optionally containing a heteroatom, the heteroatom being an oxygen or a nitrogen substituted with R⁹;
Q is selected from the group consisting of aryl, heteroaryl, and a 3-7 membered saturated ring optionally containing a heteroatom, the heteroatom being an oxygen or a nitrogen substituted with R⁹;
R⁹ is H, C₁₋₆alkyl, or C₁₋₆alkoxy-C₁₋₆alkyl;
p is an integer of 0, 1, 2, or 3;
Z is CH₂ or C(═O).

2. The compound of claim 1, wherein the carboxylic acid bioisosters are selected from the group consisting of —P(═O)(OH)₂, —C(═O)NHOH, C(═O)NHCN, 1,2,4-oxadiazol-5(4H)-one, and 3-hydroxy-4-methylcyclobut-3-ene-1,2-dione.

3. The compound of claim 1, wherein R⁴ is methyl, or ethyl.

4. The compound of claim 1, wherein R⁵ is thiazolyl.

5. The compound of claim 1, wherein X is C(═O).

6. The compound of claim 1, wherein R⁶ is C₁₋₆alkyl.

7. The compound of claim 1, wherein m is 1, n is 0 and r is 1.

8. The compound of claim 1, wherein Z is CH₂.

9. The compound of claim 1, wherein R⁷ is C₁₋₆alkyl substituted with —COOH.

10. The compound of claim 1, wherein R⁷ is (CH₂)ₚ-Q-CO₂H.

11. The compound according to claim 1, wherein Q is phenyl.

12. The compound according to claim 1, wherein Q is a C₃₋₆ cycloalkyl.

13. The compound according to claim 1, wherein Q is a 3-to 6-saturated membered ring containing an oxygen.

14. The compound according to claim 1, selected from the group consisting of the compounds having the following formulae:

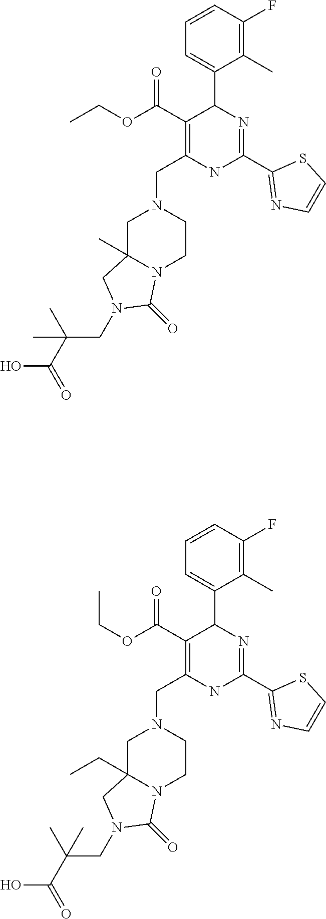 [SMILES: CCOC(=O)C1=C(CN2CCN3C(=O)N(CC(C)(C)C(=O)O)CC3(C)C2)NC(c2nccs2)=NC1c1cccc(F)c1C ; CCOC(=O)C1=C(CN2CCN3C(=O)N(CC(C)(C)C(=O)O)CC3(CC)C2)NC(c2nccs2)=NC1c1cccc(F)c1C]

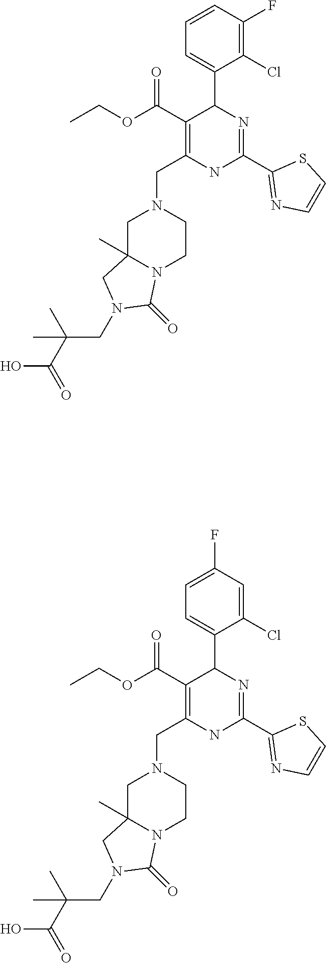 [SMILES: CCOC(=O)C1=C(CN2CCN3C(=O)N(CC(C)(C)C(=O)O)CC3(C)C2)NC(c2nccs2)=NC1c1cccc(F)c1Cl ; CCOC(=O)C1=C(CN2CCN3C(=O)N(CC(C)(C)C(=O)O)CC3(C)C2)NC(c2nccs2)=NC1c1ccc(F)cc1Cl]

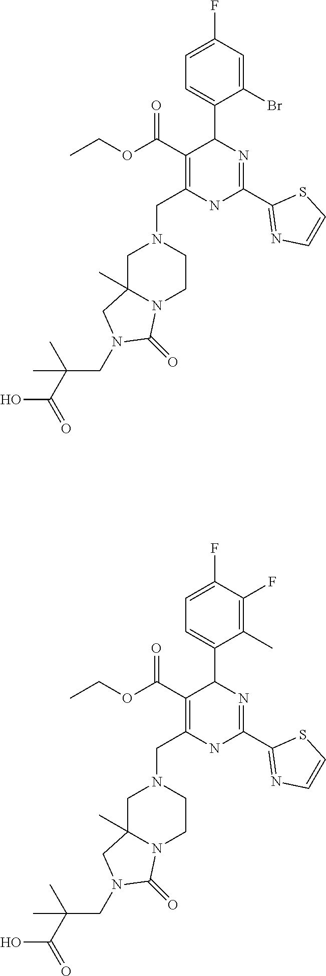 [SMILES: CCOC(=O)C1=C(CN2CCN3C(=O)N(CC(C)(C)C(=O)O)CC3(C)C2)NC(c2nccs2)=NC1c1ccc(F)cc1Br ; CCOC(=O)C1=C(CN2CCN3C(=O)N(CC(C)(C)C(=O)O)CC3(C)C2)NC(c2nccs2)=NC1c1ccc(F)c(F)c1C]

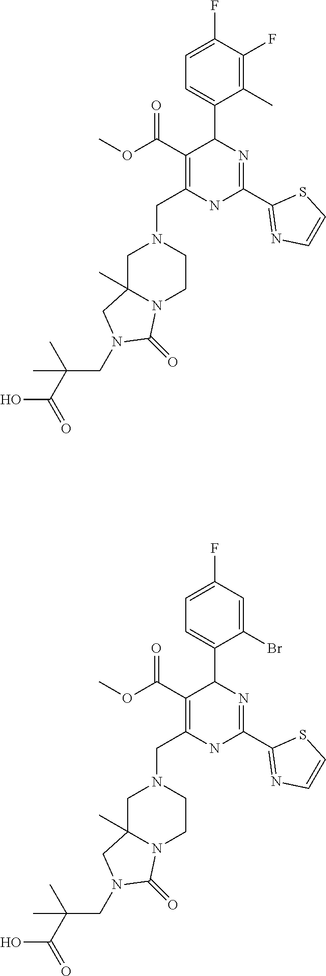 [SMILES: COC(=O)C1=C(CN2CCN3C(=O)N(CC(C)(C)C(=O)O)CC3(C)C2)NC(c2nccs2)=NC1c1ccc(F)c(F)c1C ; COC(=O)C1=C(CN2CCN3C(=O)N(CC(C)(C)C(=O)O)CC3(C)C2)NC(c2nccs2)=NC1c1ccc(F)cc1Br]

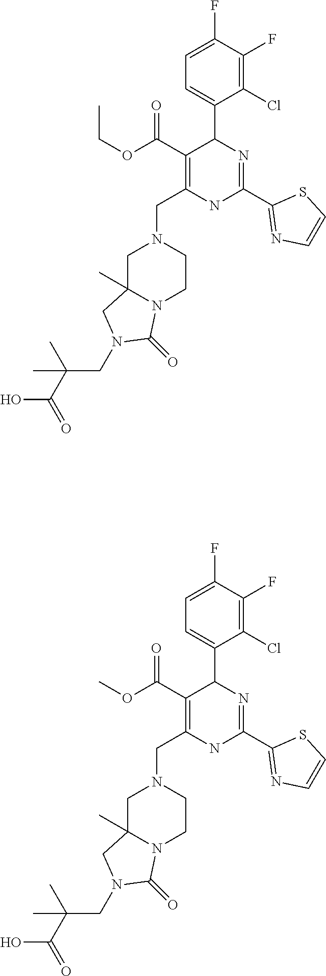 [SMILES: CCOC(=O)C1=C(CN2CCN3C(=O)N(CC(C)(C)C(=O)O)CC3(C)C2)NC(c2nccs2)=NC1c1ccc(F)c(F)c1Cl ; COC(=O)C1=C(CN2CCN3C(=O)N(CC(C)(C)C(=O)O)CC3(C)C2)NC(c2nccs2)=NC1c1ccc(F)c(F)c1Cl]

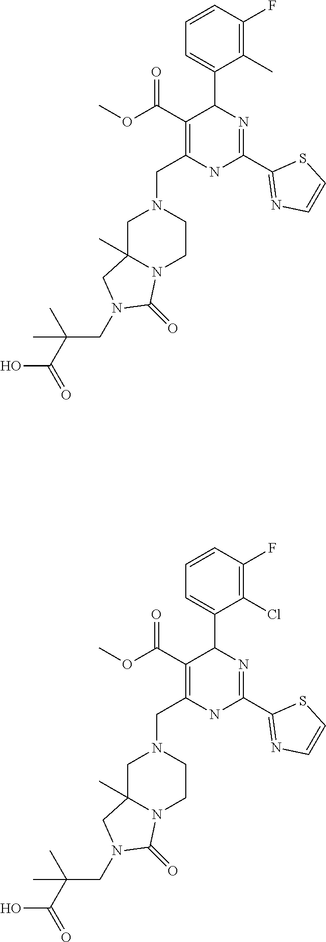 [SMILES: COC(=O)C1=C(CN2CCN3C(=O)N(CC(C)(C)C(=O)O)CC3(C)C2)NC(c2nccs2)=NC1c1cccc(F)c1C ; COC(=O)C1=C(CN2CCN3C(=O)N(CC(C)(C)C(=O)O)CC3(C)C2)NC(c2nccs2)=NC1c1cccc(F)c1Cl]

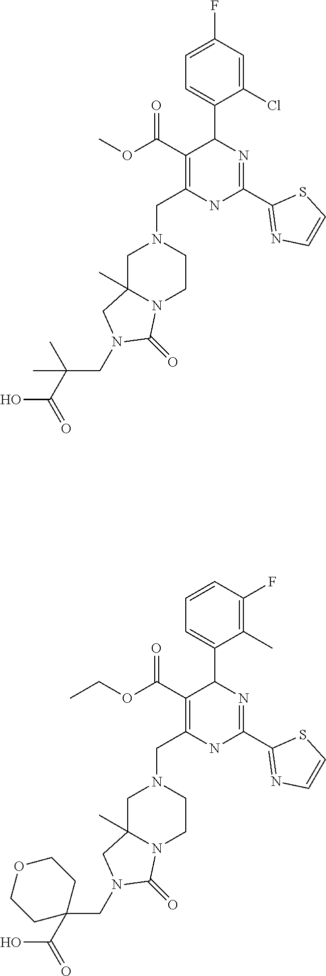 [SMILES: COC(=O)C1=C(CN2CCN3C(=O)N(CC(C)(C)C(=O)O)CC3(C)C2)NC(c2nccs2)=NC1c1ccc(F)cc1Cl ; CCOC(=O)C1=C(CN2CCN3C(=O)N(CC4(C(=O)O)CCOCC4)CC3(C)C2)NC(c2nccs2)=NC1c1cccc(F)c1C]

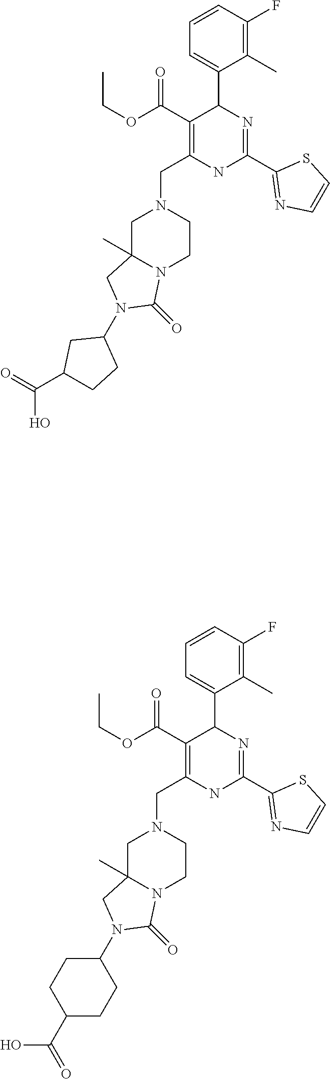 [SMILES: CCOC(=O)C1=C(CN2CCN3C(=O)N(C4CCC(C(=O)O)C4)CC3(C)C2)NC(c2nccs2)=NC1c1cccc(F)c1C ; CCOC(=O)C1=C(CN2CCN3C(=O)N(C4CCC(C(=O)O)CC4)CC3(C)C2)NC(c2nccs2)=NC1c1cccc(F)c1C]

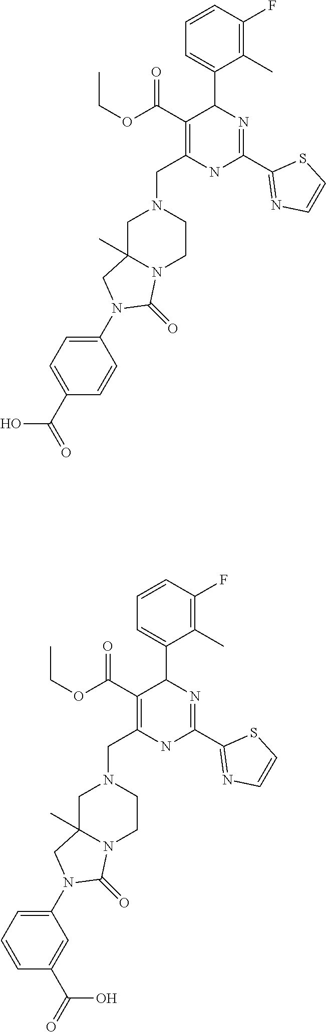 [SMILES: CCOC(=O)C1=C(CN2CCN3C(=O)N(c4ccc(C(=O)O)cc4)CC3(C)C2)NC(c2nccs2)=NC1c1cccc(F)c1C ; CCOC(=O)C1=C(CN2CCN3C(=O)N(c4cccc(C(=O)O)c4)CC3(C)C2)NC(c2nccs2)=NC1c1cccc(F)c1C]

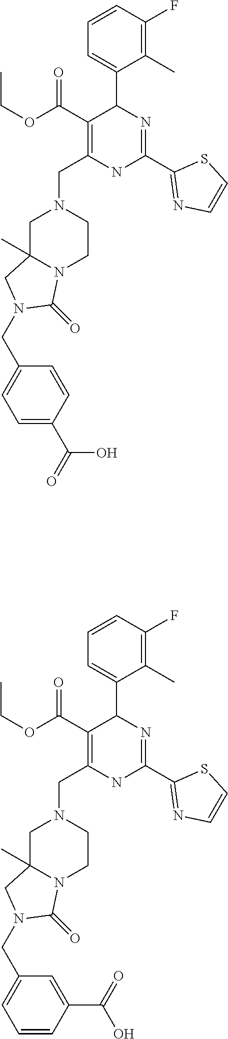 [SMILES: CCOC(=O)C1=C(CN2CCN3C(=O)N(Cc4ccc(C(=O)O)cc4)CC3(C)C2)NC(c2nccs2)=NC1c1cccc(F)c1C ; CCOC(=O)C1=C(CN2CCN3C(=O)N(Cc4cccc(C(=O)O)c4)CC3(C)C2)NC(c2nccs2)=NC1c1cccc(F)c1C]

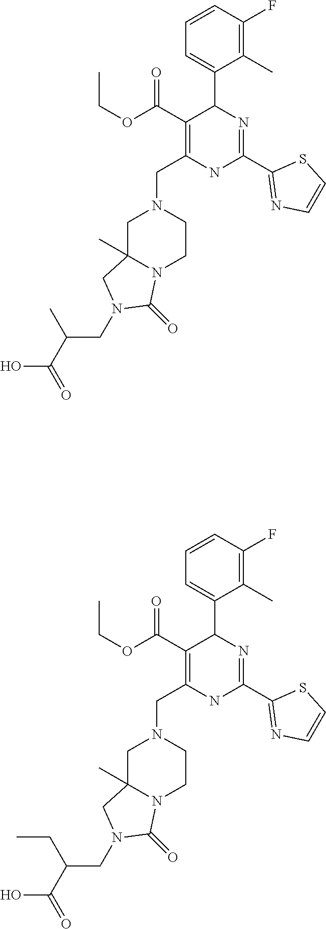 [SMILES: CCOC(=O)C1=C(CN2CCN3C(=O)N(CC(C)C(=O)O)CC3(C)C2)NC(c2nccs2)=NC1c1cccc(F)c1C ; CCOC(=O)C1=C(CN2CCN3C(=O)N(CC(CC)C(=O)O)CC3(C)C2)NC(c2nccs2)=NC1c1cccc(F)c1C]

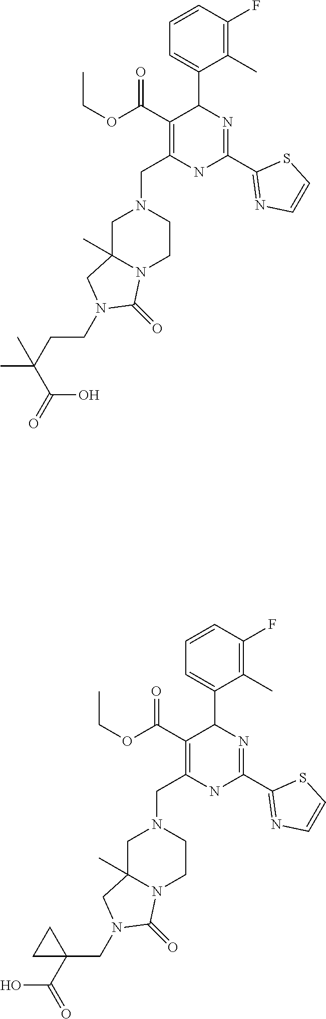 [SMILES: CCOC(=O)C1=C(CN2CCN3C(=O)N(CCC(C)(C)C(=O)O)CC3(C)C2)NC(c2nccs2)=NC1c1cccc(F)c1C ; CCOC(=O)C1=C(CN2CCN3C(=O)N(CC4(C(=O)O)CC4)CC3(C)C2)NC(c2nccs2)=NC1c1cccc(F)c1C]

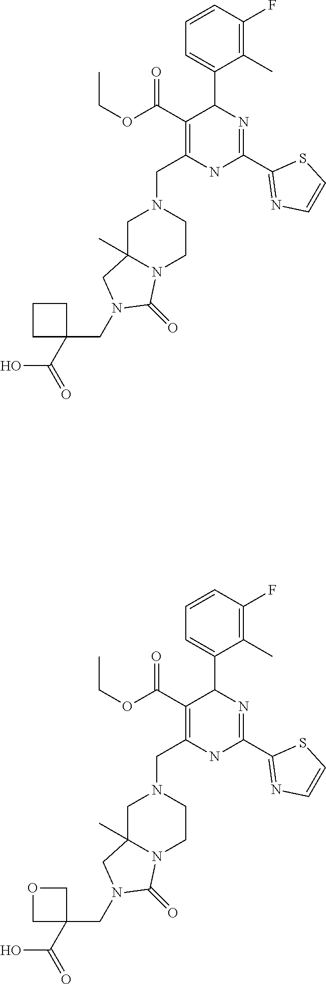 [SMILES: CCOC(=O)C1=C(CN2CCN3C(=O)N(CC4(C(=O)O)CCC4)CC3(C)C2)NC(c2nccs2)=NC1c1cccc(F)c1C ; CCOC(=O)C1=C(CN2CCN3C(=O)N(CC4(C(=O)O)COC4)CC3(C)C2)NC(c2nccs2)=NC1c1cccc(F)c1C]

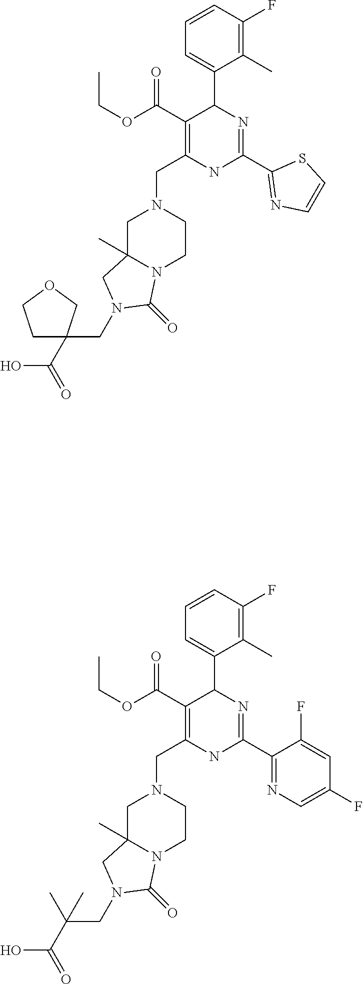 [SMILES: CCOC(=O)C1=C(CN2CCN3C(=O)N(CC4(C(=O)O)CCOC4)CC3(C)C2)NC(c2nccs2)=NC1c1cccc(F)c1C ; CCOC(=O)C1=C(CN2CCN3C(=O)N(CC(C)(C)C(=O)O)CC3(C)C2)NC(c2ncc(F)cc2F)=NC1c1cccc(F)c1C]

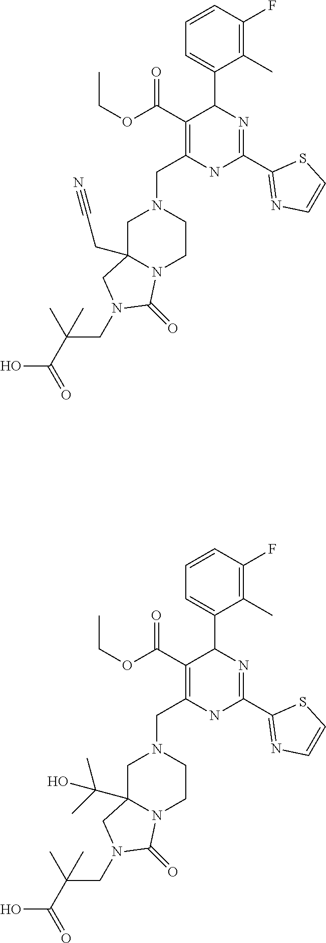 [SMILES: CCOC(=O)C1=C(CN2CCN3C(=O)N(CC(C)(C)C(=O)O)CC3(CC#N)C2)NC(c2nccs2)=NC1c1cccc(F)c1C ; CCOC(=O)C1=C(CN2CCN3C(=O)N(CC(C)(C)C(=O)O)CC3(C(C)(C)O)C2)NC(c2nccs2)=NC1c1cccc(F)c1C]

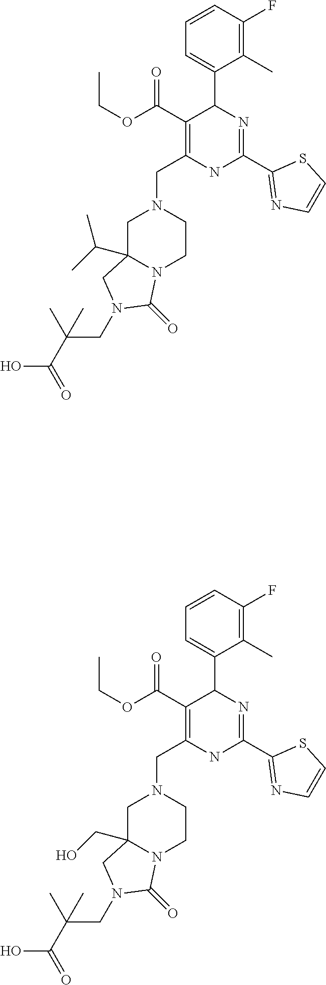 [SMILES: CCOC(=O)C1=C(CN2CCN3C(=O)N(CC(C)(C)C(=O)O)CC3(C(C)C)C2)NC(c2nccs2)=NC1c1cccc(F)c1C ; CCOC(=O)C1=C(CN2CCN3C(=O)N(CC(C)(C)C(=O)O)CC3(CO)C2)NC(c2nccs2)=NC1c1cccc(F)c1C]

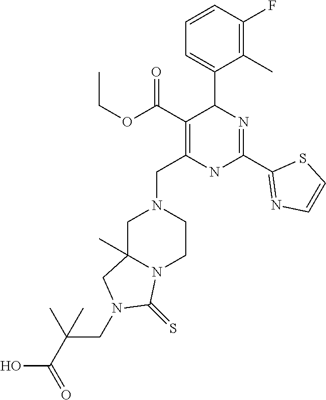 [SMILES: CCOC(=O)C1=C(CN2CCN3C(=S)N(CC(C)(C)C(=O)O)CC3(C)C2)NC(c2nccs2)=NC1c1cccc(F)c1C]

15. A pharmaceutical composition, which comprises the compound of claim 1 and which further comprises at least one pharmaceutically acceptable carrier.

16. The compound or pharmaceutically acceptable salt of claim 1, for use as a medicament.

17. The compound or pharmaceutically acceptable salt of claim 1, for use in the prevention or treatment of an HBV infection or of an HBV-induced disease in mammal in need thereof.

18. The compound or pharmaceutically acceptable salt of claim 1, for use in the prevention or treatment of chronic Hepatitis B.

19. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound or pharmaceutically acceptable salt of claim 1, and wherein said second compound is another HBV inhibitor which is selected from the group consisting of HBV combination drugs, HBV DNA polymerase inhibitors, immunomodulators, toll-like (TLR) receptor modulators, interferon alpha receptor ligands, hyaluronidase inhibitors, hepatitis b surface antigen (HbsAg) inhibitors, cytotoxic T-lymphocyte-associated protein 4 (ipi4) inhibitors, cyclohilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, farnesoid X receptor agonists, HBV antibodies, CCR2 chemokine antagonists, thymosin agonists, cytokines, nucleoprotein modulators, retinoic acid-inducible gene 1 stimulators, NOD2 stimulators, phosphatidylinositol 3-kinase (P13K) inhibitors, indoleamine 2,3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and anti-HBV drugs.

20. A process for the preparation of a compound according to claim 1, comprising the steps of:
a. The condensation of aldehyde of Formula (II), wherein Formula (II) is 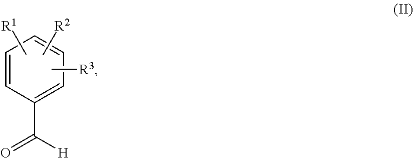
acetoacetate of Formula (III), wherein Formula (III) is 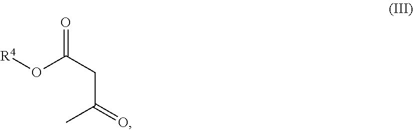
and amidine of Formula (IV), wherein Formula (IV) is 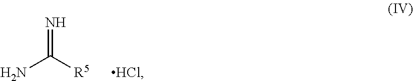
in the presence of a base, the base being preferably NaOAc, to form a compound according to Formula (I-1): 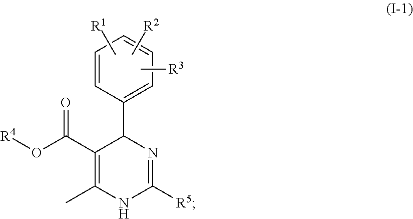
b. The bromination of compound of Formula (I-1), the brominating agent being preferably N-Bromosuccinimide, to form a compound according to Formula (I-2), wherein Formula (I-2) is 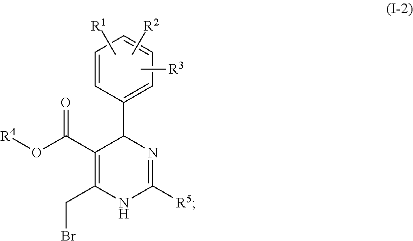
c. The coupling of compound of Formula (I-2) with a compound of Formula (V), wherein Formula (V) is 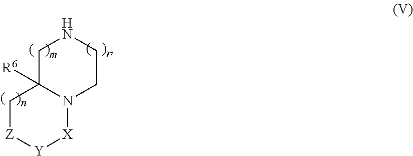
in the presence of a base, the base being preferably triethanolamine, to form a compound according to Formula (I).

21. A compound having the following structure, including any salts thereof

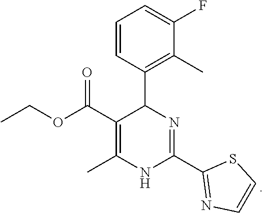 [SMILES: CCOC(=O)C1=C(C)NC(c2nccs2)=NC1c1cccc(F)c1C]

22. A compound having the following structure, including any salts thereof: 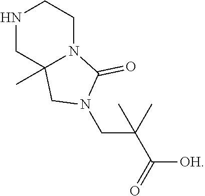 [SMILES: CC(C)(CN1CC2(C)CNCCN2C1=O)C(=O)O]


SEQUENCE LISTING (3 sequences)

SEQ ID NO: 1 - DNA, 20 bp, Artificial
  primer
gtgtctgcggcgttttatca

SEQ ID NO: 2 - DNA, 21 bp, Artificial
  primer
gacaaacgggcaacatacctt

SEQ ID NO: 3 - DNA, 28 bp, Artificial
  probe
cctctkcatcctgctgctatgcctcatc